(19) United States
(12) Patent Application Publication
(10) Pub. No.: US 2026/0295290 A1
(43) Pub. Date: Oct. 1, 2026

(54) PHOTOTHERAPY DEVICE WITH TOUCH-BASED ZONE SELECTION AND AI-DRIVEN SKIN ANALYSIS

(71) Applicant: Shenzhen Kaiyan Medical Equipment Co., Ltd., Shenzhen (CN)

(72) Inventors: Alain Dijkstra, Amstelveen (NL); Chen Shoufeng, Shenzhen (CN)

(73) Assignee: Shenzhen Kaiyan Medical Equipment Co., Ltd., Shenzhen (CN)

(21) Appl. No.: 19/577,415

(22) Filed: Mar. 25, 2026

(30) Foreign Application Priority Data
Mar. 26, 2025 (CN) 202510371838X
Mar. 26, 2025 (CN) 2025103776370

(51) Int. Cl. A61N 5/06; G06F 3/041; G06F 3/0484; G06T 7/00; G06V 10/25; G06V 40/10

(52) U.S. Cl. CPC A61N 5/0616; G06F 3/0416; G06T 7/0012; G06V 10/25; G06V 40/10; A61N 2005/0626; A61N 2005/0647; A61N 2005/0652; A61N 2005/0653; G06F 3/0484; G06F 2203/04105; G06T 2207/30088; G06V 2201/03

(57) ABSTRACT

Embodiments of the present invention disclose a phototherapy device system comprising a mask body with a multi-layer construction including a touch-sensitive layer, a backlight layer, a flexible light panel carrying LED beads, and an inner translucent bottom layer. In a touch-based mode, a touch trajectory determination module determines touch trajectories from user gestures on the touch-sensitive layer, a touch operation type determination module classifies the gestures as single-point, trajectory, or region selections, and a mask area determination module determines corresponding treatment zones. In an AI-based mode, an imaging device captures facial images processed by a pretrained skin blemish recognition model to detect blemish regions and types, which are mapped to target LED sets on the mask body. A phototherapy operation control module selectively activates LED beads in determined treatment zones according to phototherapy regimens based on blemish type, severity, or user selection.

### TECHNICAL FIELD

The present disclosure relates to wearable phototherapy devices and user interfaces therefor, and more particularly to a flexible phototherapy mask that integrates touch-based zone selection on a touch-sensitive mask surface with AI-driven skin analysis for automatic detection and classification of skin blemishes, enabling targeted LED phototherapy delivery to user-selected or automatically identified treatment regions.

### BACKGROUND ART

Phototherapy using light-emitting diodes has become a well-established modality in dermatology for treating various skin conditions, including acne, wound healing, actinic keratosis, and psoriasis, with an excellent safety profile. Phototherapy face masks have grown popular for at-home skin therapy, offering non-invasive treatment modes using red, blue, infrared, and other wavelengths that address signs of aging, acne, and other concerns. The global market for LED facial masks is rapidly expanding, reflecting advances in flexible electronics, the desire for home-use medical devices, and consumer demand for personalized skincare technology.

Various approaches have been developed in the prior art to address phototherapy device control and user interaction. Touch devices with light-emitting functions have been proposed that utilize a sensing layer to obtain touch data and force data of touch objects, where a control unit changes the emitting modes of a light-emitting module based on the touch data and force data to provide visual feedback regarding input operations. Such devices may adjust brightness, blinking frequency, or display light bars corresponding to touch gestures. However, these touch devices with light-emitting functions may be designed primarily as general input interfaces for electronic devices rather than specifically for phototherapy zone selection on wearable facial masks.

Electronic devices have been proposed that determine skin information from facial images obtained via a camera and generate control information for controlling a separate skin-care device to perform care based on the determined skin information and information regarding the skin-care device. Such electronic devices may analyze a user's skin condition from a captured image and provide control information to a mask-type LED device. However, these systems may require the user to operate multiple separate components, as the skin analysis is performed on an external electronic device rather than being integrated with the phototherapy mask itself. The skin-care devices controlled by such systems may be operated in preset operation modes, performing skin care according to one mode selected from preset operation modes, without providing direct on-mask interaction for zone selection.

Skin care systems have been proposed that employ smartphone applications to measure a user's skin condition from captured images and then set LED wavelengths accordingly, transmitting the configuration to a separate mask device via a controller. Such systems may use an application installed on a smartphone to capture facial skin images, measure skin condition parameters such as skin tone, moisture level, and elasticity, and transmit the measured skin condition information to a controller that sets or controls the wavelength of LEDs applied from the mask. However, these smartphone-based systems may require the user to operate a separate smartphone application before each treatment session and may not provide seamless integration between the skin analysis and the mask itself.

App-based platform systems have been developed that use facial skin scanners to generate facial skin map information, generate facial skin diagnosis map information, and create LED irradiation recipe mode information, including illumination group information and wavelength band information to be output by skin irradiation time. Such systems may store information on cloud servers and provide treatment protocols through smart device applications. The generated information may be provided to doctor terminals for secondary modification before being transmitted to the mask device. However, these app-based platform systems may require complex infrastructure, including facial scanners, cloud servers, and doctor terminals, and may require clinician involvement or manual configuration steps before treatment can proceed. Such systems may not allow direct, intuitive on-mask zone selection through simple touch gestures.

Beauty instruments have been developed that monitor action duration on skin areas and provide prompts to the user regarding treatment timing. Such instruments may detect whether the beauty instrument is in contact with a skin area, track the duration of continuous action on that area, and issue prompt information when the action duration reaches a preset threshold to prevent skin damage from overexposure. However, these beauty instruments may focus on timing control for handheld devices rather than providing zone-selective phototherapy on wearable facial masks.

Despite these developments, existing phototherapy masks may lack intuitive zone selection controls that allow users to directly interact with the mask surface to define treatment areas. Prior devices may apply uniform lighting over the entire mask or switch between fixed modes via buttons, without allowing the user to easily target specific facial areas through direct touch interaction. Users may desire to treat localized regions, such as a single blemish, one side of the face, or custom-drawn areas, but existing systems may rely on menu-driven interfaces, button operations, or external device configuration to select treatment zones. Furthermore, prior approaches may fail to distinguish between different touch operation types, such as single-point selection, trajectory selection, and area selection, thereby limiting the precision and flexibility of zone targeting. Existing approaches may also lack automatic mapping of detected blemishes directly to corresponding LED clusters on the mask surface, and may not provide an integrated touch interface on the mask itself for intuitive zone selection and control.

Accordingly, there is a desire for phototherapy mask systems that incorporate AI-driven skin analysis to automatically detect blemishes, map them to corresponding LEDs on the mask, select appropriate therapy regimens, and provide an on-mask touch interface for interactive control without requiring external devices or manual configuration.

### OBJECTS OF THE INVENTION

Some of the objects of the invention are as follows:

An object of the present disclosure is to provide a phototherapy device system that integrates touch-based zone selection with AI-driven skin analysis for targeted LED phototherapy delivery.

Another object of the present disclosure is to provide a phototherapy mask enabling intuitive, gesture-based selection of treatment regions on a flexible touch-sensitive mask surface.

Yet another object of the present disclosure is to allow a user to select a localized region by tapping once on the mask, such as single-point selection, and to define a treatment area around that point.

A further object of the present disclosure is to allow a user to select a contiguous path region by drawing an open curve on the mask, such as a trajectory selection.

Still another object of the present disclosure is to allow a user to select an enclosed region by drawing a closed loop on the mask, such as area selection, thereby automatically selecting the interior area.

An object of the present disclosure is to determine the specific mask area corresponding to the detected touch trajectory according to the touch operation type.

Another object of the present disclosure is to adapt the width of the selected area based on the touch force applied during the gesture.

Yet another object of the present disclosure is to personalize the reference touch width for different users, different fingers, or different mask regions so that selection feels natural.

A further object of the present disclosure is to provide immediate visual feedback of the selected mask area through indicator lights on the mask and/or on a connected display interface.

Still another object of the present disclosure is to allow the user to adjust the selected area before treatment using gestures to grow, shrink, reshape, or redraw the boundary of the region.

An object of the present disclosure is to allow the user to confirm or cancel the selected area by a simple action, such as double-tapping inside the area to confirm or double-tapping outside to cancel.

Another object of the present disclosure is to automatically determine the phototherapy mode based on the input gesture, finger identity, or instrument used.

Yet another object of the present disclosure is to detect the type of topical phototherapy agent applied to the skin in the selected area and switch to the optimal light wavelength for that substance.

A further object of the present disclosure is to provide an AI phototherapy mask system that automatically analyzes facial images to identify the locations and types of skin blemishes using a pretrained AI model.

Still another object of the present disclosure is to enable the detection of multiple dermatological conditions, including acne, eczema, freckles, and hyperpigmentation, and to estimate their severity levels.

An object of the present disclosure is to determine, for each detected blemish region, the corresponding set of LED light sources on the phototherapy mask that overlap with that facial area.

Another object of the present disclosure is to select, from a predefined mapping, an appropriate LED phototherapy regimen for each blemish type.

Yet another object of the present disclosure is to actuate the phototherapy mask's LEDs to deliver targeted light therapy only to the identified blemish regions, without requiring the user to manually select zones.

A further object of the present disclosure is to provide a user interface including touch input on the phototherapy mask or a display that allows the user or operator to review and, if needed, correct the automatically detected blemish regions and types before treatment.

Still another object of the present disclosure is to adapt the treatment parameters based on patient-specific factors such as skin tone, previous treatment history, and lesion severity.

An object of the present disclosure is to integrate sensing features such as capacitive touch feedback, fit sensors, and 3D face mapping to ensure the phototherapy mask aligns properly with the face and to allow intuitive zone selection.

Another object of the present disclosure is to give the user visual feedback of the active treatment areas and enable direct controls on the phototherapy mask.

Yet another object of the present disclosure is to enable wireless connectivity so that treatment sessions can be uploaded to a cloud server or external device for remote review, monitoring, and software updates.

A further object of the present disclosure is to control the LEDs within the defined mask area to perform phototherapy in the chosen mode, thereby improving efficiency and enabling different regions to be treated independently.

Still another object of the present disclosure is to improve the precision, consistency, and overall efficacy of phototherapy by targeting only the needed regions with the appropriate light, thereby reducing reliance on operator experience and improving the user experience.

### SUMMARY OF THE INVENTION

According to a first aspect of the invention, a phototherapy device system is provided. The phototherapy device system comprising: a touch-sensing module configured to detect a touch operation on a surface of a phototherapy device; a trajectory determination module configured to determine a touch trajectory corresponding to the touch operation; a mask area determination module configured to determine, based on the touch trajectory, a user-selected region of the phototherapy device; a light-emitting control module configured to identify a plurality of light-emitting elements corresponding to the user-selected region; a processing module configured to determine a phototherapy parameter associated with the user-selected region; and a control module configured to control the plurality of light-emitting elements corresponding to the user-selected region to emit light according to the phototherapy parameter.

In one embodiment of the invention, the touch trajectory comprises at least one of a single-point touch, an open trajectory, or a closed trajectory; and the user-selected region comprises at least one of: a point region corresponding to the single-point touch, a path region corresponding to the open trajectory, or an enclosed region corresponding to the closed trajectory.

In one embodiment of the invention, the touch-sensing module is further configured to determine a touch force associated with the touch operation.

In one embodiment of the invention, the mask area determination module is configured to determine the size of the user-selected region based on the touch force.

In one embodiment of the invention, the processing module is configured to determine the phototherapy parameter based on user input corresponding to a selected phototherapy mode.

In one embodiment of the invention, the processing module is further configured to: acquire image data corresponding to the user-selected region; and analyze the image data to identify at least one skin characteristic, wherein the phototherapy parameter is determined based on the skin characteristic.

In one embodiment of the invention, the processing module is configured to determine a recommended phototherapy parameter based on the image data, and the phototherapy parameter is determined based on user modification of the recommended phototherapy parameter.

In one embodiment of the invention, the light-emitting control module is configured to place the plurality of light-emitting elements in a standby state before emission of light based on the phototherapy parameter.

In one embodiment of the invention, the phototherapy device comprises a plurality of touch-sensitive elements distributed across a surface of the phototherapy device, each touch-sensitive element being spatially aligned with a corresponding light-emitting element; each touch-sensitive element comprises at least one of a capacitive sensor, a resistive sensor, or a pressure sensor; and a touch detected by a given touch-sensitive element activates the corresponding light-emitting element to emit light according to the phototherapy parameter.

In one embodiment of the invention, the phototherapy device comprises a touch-sensitive layer disposed on a surface of the phototherapy device, and the touch-sensitive layer is configured to detect a spatial location of a touch input and activate a corresponding subset of the plurality of light-emitting elements to emit light according to the phototherapy parameter.

In one embodiment of the invention, the processing module is configured to: determine a severity level associated with a skin characteristic of the user-selected region, and adjust the phototherapy parameter based on the severity level, wherein the severity level is determined based on at least one of a size, intensity, density, or spatial distribution of the skin characteristic.

According to a second aspect of the invention, a method for operating a phototherapy device is provided. The method comprising: detecting a touch operation on a surface of the phototherapy device; determining a touch trajectory corresponding to the touch operation; determining, based on the touch trajectory, a user-selected region of the phototherapy device; identifying a plurality of light-emitting elements corresponding to the user-selected region; determining a phototherapy parameter associated with the user-selected region; and controlling the plurality of light-emitting elements corresponding to the user-selected region to emit light according to the phototherapy parameter.

In one embodiment of the invention, the method further comprising: acquiring image data corresponding to the user-selected region; and determining the phototherapy parameter based on the image data.

In one embodiment of the invention, the method further comprising: detecting an indication associated with the user-selected region, wherein the indication comprises at least one of a therapeutic substance, a color marking, a symbol, a pattern, or a visually or sensor-detectable marker; and determining the phototherapy parameter based on the detected indication.

In one embodiment of the invention, determining the phototherapy parameter comprises: determining the phototherapy parameter based on a type of finger used for the touch operation, and determining an intensity of emitted light based on a pressure associated with the touch operation, wherein different fingers correspond to different phototherapy parameters.

In one embodiment of the invention, the method further comprising: providing feedback indicating the user-selected region, wherein the feedback comprises at least one of: haptic feedback including vibration generated by the phototherapy device, or visual feedback including illumination of one or more light-emitting elements, and wherein the feedback is provided on at least one of an outer surface or an inner surface of the phototherapy device corresponding to the user-selected region.

In one embodiment of the invention, determining the user-selected region comprises mapping the touch trajectory to a spatial distribution of the plurality of light-emitting elements on a curved surface of the phototherapy device.

According to a third aspect of the invention, a method for operating a phototherapy device is provided. The method comprising: selecting at least one region of a user's skin via at least one user interface associated with the phototherapy device, the selected at least one region corresponding to a region of the phototherapy device; acquiring image data corresponding to the selected at least one region; processing the image data to determine at least one skin characteristic associated with the selected at least one region; determining a phototherapy parameter for the selected at least one region based on the determined at least one skin characteristic; and controlling a plurality of light-emitting elements corresponding to the selected at least one region to emit light according to the phototherapy parameter.

In one embodiment of the invention, selecting the at least one region comprises receiving a touch input on a surface of the phototherapy device.

In one embodiment of the invention, selecting the at least one region comprises selecting the at least one region from a visual representation of the user's skin displayed on an external device.

In one embodiment of the invention, the external device comprises at least one of a mobile device or a smart mirror; and a region selection input comprises at least one symbol corresponding to a selected region of the user's skin, and the phototherapy parameter is assigned to the selected region based on the symbol.

In one embodiment of the invention, selecting the at least one region comprises selecting two or more regions associated with different skin characteristics; respective phototherapy parameters correspond to different wavelengths or light characteristics; and the plurality of light-emitting elements are controlled to simultaneously emit different lights in the respective regions.

In one embodiment of the invention, processing the image data comprises generating a recommended phototherapy parameter based on the image data, and the phototherapy parameter is determined based on at least one of user confirmation or user modification of the recommended phototherapy parameter.

According to a fourth aspect of the invention, a phototherapy control system is provided. The phototherapy control system comprising: an image acquisition module configured to acquire image data of a user; a processing module configured to: identify at least one skin region and a corresponding skin characteristic from the image data, and determine a phototherapy parameter based on the skin characteristic; and a control module configured to control a plurality of light-emitting elements corresponding to the at least one skin region to emit light according to the phototherapy parameter.

In one embodiment of the invention, the processing module is further configured to identify the at least one skin region based on a user input received via at least one of: a touch input on the phototherapy device, or an input received from an external device.

In one embodiment of the invention, the processing module is configured to: determine a capture angle of the image data; and generate corrected image data based on the capture angle before identifying the at least one skin region.

In one embodiment of the invention, the processing module is configured to determine a severity level associated with the skin characteristic, and the severity level is determined based on at least one of the size, intensity, density, or spatial distribution of the skin characteristic.

In one embodiment of the invention, the system further comprises a memory configured to store historical phototherapy data, and the processing module is configured to: compare current image data with the historical phototherapy data to determine a treatment effect, and adjust the phototherapy parameter based on the treatment effect.

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second,” and “third” are only used for descriptive purposes and do not imply the relative importance or implicitly indicate the quantity of technical features indicated.

In the context of the specification, the term “plurality” means two or more than two, unless otherwise indicated.

In the context of the specification, the term “phototherapy element” encompasses any light-emitting device capable of emitting light of therapeutic wavelength(s), including but not limited to light-emitting diodes (LEDs), organic LEDs (OLEDs), laser diodes, or equivalent optical sources. The light may include ultraviolet, visible, near-infrared, or far-infrared spectra.

In the context of the present disclosure, the term “phototherapy parameter” refers to a treatment setting that controls the characteristics of light emitted by phototherapy elements during a treatment session. A phototherapy parameter may comprise at least one of wavelength, intensity, duration, or duty cycle. Wavelength refers to the spectral characteristic of the emitted light, typically measured in nanometers, and may include visible light wavelengths such as red light (approximately 620-750 nm), blue light (approximately 415-470 nm), green light (approximately 520-540 nm), and yellow light (approximately 580-600 nm), as well as near-infrared wavelengths (approximately 750-1400 nm). Intensity refers to the power or brightness of the emitted light, which may be expressed in milliwatts per square centimeter or as a percentage of maximum output. Duration refers to the length of time during which phototherapy light is applied to a treatment zone, typically measured in seconds or minutes. Duty cycle refers to the ratio or percentage of time during which a light-emitting element is in an active or on state relative to the total period of a pulse cycle, and may be expressed as a percentage.

In the context of the present disclosure, the term “touch trajectory” refers to a path or pattern formed by one or more touch points detected on a touch-sensitive surface over time. A touch trajectory may include the sequence of coordinates corresponding to the movement of a finger, stylus, or other touch object across the surface. The touch trajectory may be formed by continuously detecting and recording the coordinates of each touch point in chronological order as the user interacts with the touch-sensitive surface.

In the context of the present disclosure, the term “touch operation type” refers to a classification or category of touch gesture determined based on characteristics of a touch trajectory. Touch operation types may include a single-point selection type corresponding to a single touch point, a trajectory selection type corresponding to an open trajectory, and a region selection type corresponding to a closed trajectory. The touch operation type may determine how the corresponding mask area is calculated from the touch trajectory.

In the context of the present disclosure, the term “mask area” refers to a region or zone on the phototherapy device corresponding to a touch trajectory or detected skin condition. A mask area may encompass one or more phototherapy light elements designated for phototherapy treatment. The mask area may be determined based on the touch operation type and may include a point region, a path region, or an enclosed region, depending on the nature of the touch trajectory.

In the context of the present disclosure, the term “skin blemish” refers to a visible or detectable abnormality, imperfection, or condition on the skin surface. Skin blemishes may include acne, pimples, eczema, freckles, hyperpigmentation, age spots, scars, rosacea, inflammation, lesions, and other dermatological conditions. Skin blemishes may be detected through analysis of facial images captured by an imaging device.

In the context of the present disclosure, the term “blemish type” refers to a classification or category assigned to a detected skin blemish based on characteristics of the blemish. Blemish types may include acne, eczema, freckles, hyperpigmentation, and other dermatological condition categories. The blemish type may be determined by a pretrained skin blemish recognition model and may be used to select an appropriate phototherapy regimen.

In the context of the present disclosure, the term “severity level” refers to a quantitative or qualitative measure indicating the intensity, extent, or seriousness of a detected skin blemish. A severity level may be expressed as mild, moderate, or severe, or as a numerical score. The severity level may be used to adjust phototherapy parameters such as treatment duration or light intensity.

In the context of the present disclosure, the term “phototherapy regimen” refers to a set of treatment parameters for phototherapy. A phototherapy regimen may include light wavelength, light intensity, treatment duration, treatment frequency, and duty cycle. The phototherapy regimen may be selected based on blemish type and severity level, or may be selected by the user through touch input or other user interface mechanisms.

In the context of the present disclosure, the term “facial landmark” refers to an anatomically significant point or feature on the face used for alignment, calibration, or reference purposes. Facial landmarks may include eye corners, nose tip, nose bridge, mouth corners, jawline points, and forehead boundaries. Facial landmarks may be detected in facial images and used to establish correspondence between facial coordinates and mask coordinates.

In the context of the specification, the term “control board or circuit board” encompasses any printed circuit board (PCB), flexible circuit, or equivalent substrate that supports and electrically connects components of the device, including power supplies, control chips, drivers, or stimulation elements.

In the context of the specification, the term “user” or “subject” is intended to broadly cover humans, animals, or other recipients of the treatment, unless otherwise specifically limited.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or Super luminous diodes (“SLD”).

In the context of the specification, the terms, such as “Light-Emitting Diodes (LEDs)”, “LED beads”, “light-emitting elements” are used interchangeably and refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In addition to conventional mineral-based LEDs, one or more LEDs may also be provided on an Organic LED (OLED) based flexible panel or an inorganic LED-based flexible panel. Such OLED panels may be generated by depositing organic semiconducting materials over Thin Film Transistor (TFT) based substrates. Further, a discussion on the generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety by reference.

In the context of this specification, terms like “light”, “radiation”, “irradiation”, “emission” and “illumination”, etc. refer to electromagnetic radiation in frequency ranges varying from the Ultraviolet (UV) frequencies to Infrared (IR) frequencies and wavelengths, wherein the range is inclusive of visible light, UV and IR frequencies and wavelengths. It is to be noted here that UV radiation can be categorized in several ways depending on respective wavelength ranges, all of which are envisaged to be under the scope of this invention. For example, UV radiation can be categorized as Hydrogen Lyman-α (122-121 nm), Far UV (200-122 nm), Middle UV (300-200 nm), and Near UV (400-300 nm). The UV radiation may also be categorized as UVA (400-315 nm), UVB (315-280 nm), and UVC (280-100 nm). Similarly, IR radiation may also be categorized into several categories according to respective wavelength ranges, which are again envisaged to be within the scope of this invention. A commonly used subdivision scheme for IR radiation includes Near IR (0.75-1.4 m), Short-Wavelength IR (1.4-3 μm), Mid-Wavelength IR (3-8 μm), Long-Wavelength IR (8-15 μm), and Far IR (15-1000 μm).

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

In an embodiment, the phototherapy unit emits multiple therapeutic wavelengths adapted for skin care and dermatological treatment. Red light (approximately 630-660 nm) penetrates deeply into the skin to stimulate blood circulation, enhance collagen production, and promote skin regeneration and repair. Blue light (around 415-470 nm) exhibits antibacterial properties and is effective in treating acne, minimizing breakouts, and reducing inflammation. Green light (approximately 520-540 nm) helps reduce hyperpigmentation, even skin tone, and soothe sensitive or irritated skin. Yellow light (around 580-600 nm) improves cellular oxygen exchange, enhances lymphatic circulation, and supports detoxification and skin revitalization. Near-infrared light (800-850 nm) penetrates deeper tissue layers to accelerate healing, reduce pain, and alleviate inflammation, thereby supporting overall skin recovery and rejuvenation.

### BRIEF DESCRIPTION OF THE ACCOMPANYING DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings, wherein like reference letters and numerals indicate the corresponding parts in various figures in the accompanying drawings, and in which:

FIG. 1 illustrates a perspective view of a phototherapy mask system, in accordance with an embodiment of the present invention.

FIG. 2 illustrates a perspective view of a mask body, in accordance with an embodiment of the present invention.

FIG. 3 illustrates an exploded view of mask layers and a controller, in accordance with an embodiment of the present invention.

FIG. 4 illustrates a block diagram of a controller including a processor and memory, in accordance with an embodiment of the present invention.

FIG. 5 illustrates a single-point touch selection on a mask surface, in accordance with an embodiment of the present invention.

FIG. 6 illustrates an open trajectory touch selection on a mask surface, in accordance with an embodiment of the present invention.

FIG. 7 illustrates a closed trajectory touch selection on a mask surface, in accordance with an embodiment of the present invention.

FIG. 8 illustrates a flowchart of a touch control method, in accordance with an embodiment of the present invention.

FIG. 9 illustrates a block diagram of a controller, in accordance with an embodiment of the present invention.

FIG. 10 illustrates a flowchart of an AI control method, in accordance with an embodiment of the present invention.

FIG. 11 illustrates a skin blemish recognition model architecture, in accordance with an embodiment of the present invention.

FIG. 12 illustrates skin blemish detection results on a face, in accordance with an embodiment of the present invention.

FIG. 13 illustrates face-to-mask LED mapping, in accordance with an embodiment of the present invention.

FIG. 14 illustrates a block diagram of an AI control device, in accordance with an embodiment of the present invention.

FIG. 15 is a flow diagram showing a method for operating a phototherapy device, in accordance with an embodiment of the present invention.

FIG. 16 is a flow diagram showing a method for operating a phototherapy device, in accordance with an embodiment of the present invention.

### DETAILED DESCRIPTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

The terms “a” and “an” herein do not denote a limitation of quantity, but rather denote the presence of at least one of the referenced items. The terms “having”, “comprising”, “including”, and variations thereof signify the presence of a component.

Embodiments of the present invention disclose a phototherapy device system that integrates touch-based zone selection with AI-driven skin analysis for targeted LED phototherapy delivery. The phototherapy device system comprises a flexible, wearable phototherapy mask configured to conform to a user's face during treatment. In some cases, the phototherapy device may comprise a phototherapy pad configured for application to other body regions. The phototherapy device system supports multiple operational modes, including touch-based selection, AI-based automatic detection, and hybrid operation combining both approaches.

The phototherapy device comprises various form factors configured for different body regions. In some cases, the phototherapy device may comprise a phototherapy mask configured to conform to the user's face for facial treatment. In some cases, the phototherapy device may comprise a phototherapy pad configured for application to other body regions such as the neck, chest, back, arms, or legs. The phototherapy pad includes the same multi-layer construction as a mask body, including a touch-sensitive layer, a flexible light panel with LED beads, and an inner translucent bottom layer, adapted to a flat or semi-flexible form factor suitable for non-facial body regions. The touch-based zone selection and AI-based skin analysis features described herein apply to both the mask and pad form factors.

In touch-based selection mode, a user directly interacts with a touch-sensitive surface of the phototherapy device to select treatment zones through various touch gestures. The touch gestures include single-point touches, open trajectory traces, and closed trajectory traces, each corresponding to different selection types for defining mask areas to receive phototherapy treatment.

In AI-based automatic detection mode, the phototherapy device system acquires facial images of the user and processes the images using a pretrained skin blemish recognition model to detect and classify skin blemish regions. The detected blemish regions are mapped to corresponding LED elements on the phototherapy device, and appropriate phototherapy regimens are selected based on blemish type and severity.

The image acquisition and subsequent image analysis or recognition are performed with the subject's knowledge and consent and in compliance with applicable requirements, such that the acquisition and processing do not impinge upon the public interest. The phototherapy device system prompts the user to provide explicit consent before capturing facial images or performing skin analysis. Consent preferences are stored in the user profile in the memory and are revocable by the user at any time.

In hybrid operation mode, the phototherapy device system combines AI-based detection with touch-based user input. The AI-based detection provides recommended treatment zones and parameters, while the touch-based input allows the user to confirm, modify, or override the AI recommendations. This hybrid approach provides both automated analysis and user control over the treatment process.

In an AI-based operational sequence, once the mask body is in place and powered on via the switch button or a touch gesture, the control sequence is largely automatic. The imaging device first captures the user's facial image. The processor runs the skin blemish recognition model to detect blemish regions and classify each region's type and severity. Based on this analysis, the processor determines the target LED clusters and selects the corresponding phototherapy regimens, including light types, intensities, and durations. The LED beads then activate accordingly, emitting therapeutic light through the inner translucent bottom layer to the skin. During treatment, the touch-sensitive layer allows user interaction; a double-tap on any active region toggles the light off in that area. A second double-tap on a region or holding the switch button for a preset time turns off the LED beads entirely to end the session. Throughout this process, the backlight layer and the inner translucent bottom layer ensure even light diffusion, and the controller manages timing and intensity as specified by the chosen regimens.

In the AI-based control method, the phototherapy device system acquires one or more facial images of the subject using an imaging device. The acquired facial images are processed using a pretrained skin blemish recognition model to detect skin blemish regions and classify each detected region with a corresponding blemish type and severity level. Each detected skin blemish region is mapped to a corresponding set of target LED beads on the mask body based on a face-to-mask coordinate mapping. A target phototherapy regimen is determined for each detected skin blemish region based on the corresponding blemish type and severity level. The target LED beads are then controlled to perform phototherapy on the subject according to the determined target phototherapy regimen.

FIG. 11 shows the skin blemish recognition model is a pretrained artificial intelligence model trained using a training dataset comprising a plurality of training samples. Each training sample includes one or more sample facial images and corresponding skin blemish recognition annotation data indicating blemish regions and blemish types. During operation, a facial image captured by the imaging device is provided as input to the trained skin blemish recognition model, which outputs detected skin blemish regions and corresponding blemish types for the input facial image. The skin blemish recognition model detects multiple distinct skin blemish regions at various locations on the subject's face, each associated with a corresponding blemish type and severity level. The number, positions, sizes, and assigned blemish types vary with individual subjects and their specific dermatological conditions.

Each detected facial blemish region is mapped to a corresponding set of target LED beads on the mask body using a stored face-to-mask coordinate transform to translate facial coordinates of each detected blemish region to corresponding mask coordinates. The AI control device comprises functional modules, including a facial image acquisition module, a skin blemish identification module, an LED bead collection determination module, a phototherapy mode determination module, and an AI phototherapy mask control module. These modules are implemented as software modules executed by the processor or as dedicated hardware circuitry within the controller.

The phototherapy device system incorporates continuous learning from user feedback and historical data to refine the skin blemish recognition model over time. Users annotate or correct the detected blemish regions and types, and the system uses this feedback to improve recognition accuracy for that individual. The system also aggregates anonymized data from users with similar skin tones and conditions to recommend the most effective phototherapy modes for the current user's skin type and blemish profile.

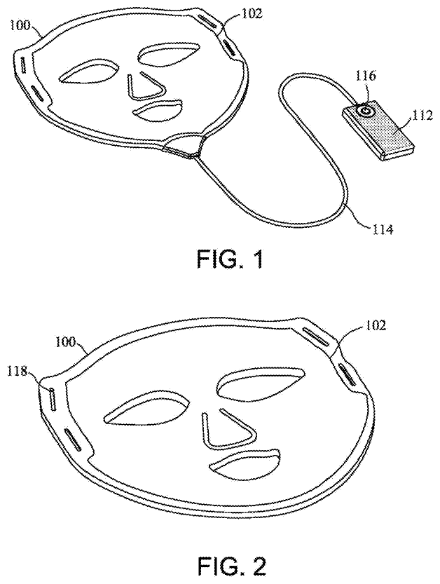

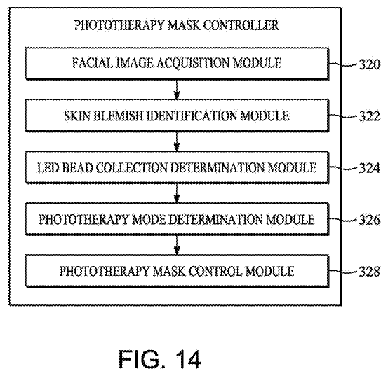

Embodiments of the present invention will now be described in detail with reference to FIGS. 1 to 16, wherein like reference numerals refer to like elements throughout.

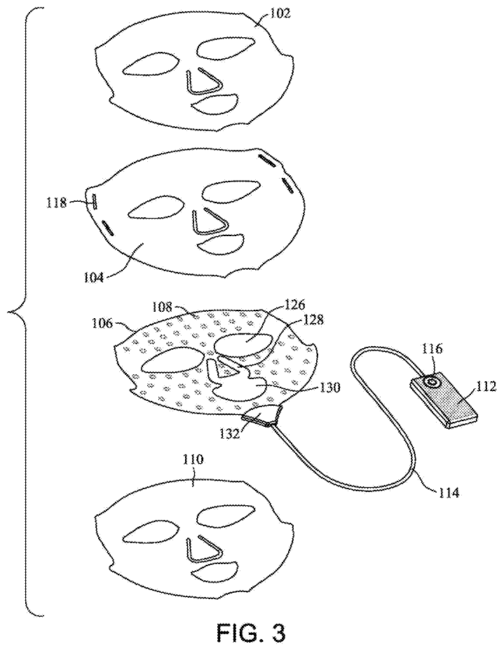

Referring to FIGS. 1-3, the phototherapy device system comprises a mask body 100 having a flexible, multi-layer construction configured to conform to the facial contours of a user during treatment. As shown in FIG. 3, the mask body 100 includes a touch-sensitive layer 102, a support layer 104, a flexible light panel 106 with LED beads 108, an inner translucent bottom layer 110, arranged in a stacked configuration, and a controller 112. The flexible, multi-layer construction allows the mask body 100 to adapt to different facial shapes and sizes while maintaining consistent contact between the inner translucent bottom layer 110 and the user's skin.

Referring to FIG. 3, the touch-sensitive layer 102 comprises a transparent capacitive touch layer configured to detect touch operations by the user on an outer surface of the mask body 100. The transparent capacitive touch layer is laminated on the outside of the support layer 104. The touch-sensitive layer 102 comprises a thin conductive mesh or indium tin oxide (ITO) coating on polyethylene terephthalate (PET) bonded to the support layer 104. The touch-sensitive layer 102 includes conductive traces crossing to form a touch grid capable of detecting touch coordinates when the user contacts the outer surface with a finger, stylus, or other touch instrument. The conductive traces of the touch-sensitive layer 102 are routed via flex traces to the controller 112 or directly to the processor 120, enabling transmission of touch coordinate data from the touch-sensitive layer 102 to the processing electronics. The touch-sensitive layer 102 is either integrated into or directly adhered to the support layer 104. In some cases, the touch-sensitive layer 102 may be physically integrated with the flexible light panel 106 and protective films within an integrated flexible layer assembly.

The support layer 104 is positioned beneath the touch-sensitive layer 102 and provides mechanical support for the mask body 100 structure. The support layer 104 provides structural rigidity while permitting controlled bending. The support layer 104 comprises a flexible polymeric material such as polyurethane that provides both structural integrity and transparency. The support layer 104 includes openings corresponding to facial features of the user.

Referring to FIG. 3, the flexible light panel 106 is positioned beneath the support layer 104 and carries an array of LED beads 108 arranged across a surface of the mask body 100. The LED beads 108 are distributed across the flexible light panel 106 in positions corresponding to various treatment regions of the face. The flexible light panel 106 comprises a flexible substrate capable of conforming to curved facial surfaces while maintaining electrical connections to the LED beads 108. The flexible light panel 106 is sandwiched between the support layer 104 and the inner translucent bottom layer 110. The flexible light panel 106 includes clearance areas on the far side of each eye opening 126 so that when straps pull the mask body 100 tight via the connecting positions 118, the flexible light panel 106 can flex without damaging the LED beads 108. The clearance areas ensure that no LED beads 108 are positioned under folded portions of the mask body 100, so that touch input and light delivery remain unaffected when the mask body 100 is tensioned onto the face.

As shown in FIG. 3, the inner translucent bottom layer 110 is positioned at the bottom of the mask body 100 stack and faces the skin of the user during use. The inner translucent bottom layer 110 is made of thermoplastic polyurethane (TPU) or polyurethane (PU) material. The inner translucent bottom layer 110 seals the LED beads 108 and forms a skin interface that contacts the user's skin during treatment. The inner translucent bottom layer 110 allows therapeutic light emitted by the LED beads 108 to pass through to the user's face while providing a comfortable contact surface. The entire multi-layer stack of the mask body 100 is sealed at the edges by welding or adhesive so that the touch-sensitive layer 102 and the flexible light panel 106 remain protected from moisture, debris, and mechanical damage.

Referring to FIGS. 2 and 3, the mask body 100 includes eye openings 126 configured to accommodate the eyes of the user during treatment. The mask body 100 includes a nose opening 128 configured to accommodate the nose of the user. The nose opening 128 includes an integrated nose therapy region surrounding the nose opening 128, where LED beads 108 are positioned to provide phototherapy treatment to the skin around the nose area. The mask body 100 includes a mouth opening 130 configured to accommodate the mouth of the user. The eye openings 126, nose opening 128, and mouth opening 130 extend through all layers of the mask body 100 to provide clearance for the corresponding facial features.

As shown in FIGS. 1 and 2, the mask body 100 includes connecting positions 118 on the sides of the mask body 100 for securing straps or bands. The connecting positions 118 comprise reinforced attachment points configured to receive adjustable straps that affix the mask body 100 to the user's head. The straps are routed through the connecting positions 118 and are tightened to hold the inner translucent bottom layer 110 snugly against the face of the user. The connecting positions 118 are located on either side of the eye openings 126 to distribute tension evenly across the mask body 100 when the straps are secured.

The phototherapy device system includes a touch-sensing module configured to detect a touch operation on a surface of the phototherapy device. The touch-sensing module is integrated into the mask body structure and continuously monitors the outer surface for user contact. The touch-sensing module converts detected touch operations into electrical signals that are transmitted to a processor for analysis and interpretation. The touch-sensing module operates in real time to provide responsive interaction between the user and the phototherapy device. The touch-sensitive layer provides a touch-sensitive surface on the outer side of the mask body through which the user performs touch operations, and the touch-sensitive layer forms the outer layer of the phototherapy device. The phototherapy device may comprise a touch-sensitive layer disposed on a surface of the phototherapy device. The touch-sensitive layer may be positioned on the outer surface of the mask body, where the user can access the touch-sensitive layer during operation. The touch-sensitive layer may extend across treatment regions of the mask body to enable zone selection across different facial areas. The touch-sensitive layer may be configured as a continuous sensing surface or may comprise discrete touch-sensitive elements distributed across the mask body surface.

The touch-sensitive layer comprises a transparent capacitive film configured to detect touch operations by the user on the outer surface of the mask body. The transparent capacitive film is laminated on the outer surface of the backlight layer to form the outermost layer of the mask body stack. The transparent capacitive film maintains optical transparency to allow visual observation of LED activation states through the mask body surface. The transparent capacitive film comprises a thin conductive layer deposited on a flexible polymer substrate that conforms to the curved contours of the mask body.

The touch-sensitive element comprises at least one of a capacitive sensor, a resistive sensor, or a pressure sensor. Capacitive sensors detect touch operations by measuring changes in capacitance when a conductive object, such as a finger, approaches or contacts the sensor surface. Resistive sensors detect touch operations by measuring changes in electrical resistance when pressure is applied to the sensor surface. Pressure sensors detect touch operations by measuring the mechanical force applied to the sensor surface and provide information about touch force in addition to touch location.

In some cases, the touch-sensitive layer may comprise capacitive sensors arranged in a grid pattern across the mask body surface. The capacitive sensors include a matrix of conductive traces oriented in perpendicular directions to form sensing nodes at intersection points. When the user contacts the touch-sensitive layer with a finger or other conductive object, the capacitive sensors detect changes in capacitance at sensing nodes near the contact location. The processor analyzes the capacitance changes across multiple sensing nodes to determine precise coordinates of the touch location.

In some cases, each of the plurality of light-emitting elements on the flexible light panel 106 may be associated with a respective touch-sensitive element disposed on the touch-sensitive layer 102. Each touch-sensitive element is spatially aligned with a corresponding light-emitting element located on an opposite side of the mask body 100, such that the touch-sensitive element on the outer surface is positioned directly above or in registration with the corresponding LED bead 108 on the flexible light panel 106. The touch-sensitive elements comprise at least one of capacitive sensors, resistive sensors, or pressure sensors. When a touch is detected by a given touch-sensitive element, the processor 120 or an internal phototherapy operation control module 216 activates the corresponding spatially aligned light-emitting element to emit light according to the phototherapy parameter. This one-to-one spatial alignment between touch-sensitive elements and light-emitting elements enables direct, intuitive zone selection where touching a specific location on the outer surface immediately identifies the corresponding LED bead 108 for activation.

Referring to FIG. 5, a single-point touch selection on the mask body 100 is illustrated. A user's finger 506 is shown making contact with the touch-sensitive layer 102 at a single touch point 500. When the touch operation type determination module 212 detects that the touch trajectory corresponds to the single touch point 500, the touch operation type is identified as a single-point selection type. The mask area determination module 214 designates a circular region centered at the single touch point 500, with a preset touch width as its diameter, as the mask area for phototherapy treatment. The LED beads 108 located within the determined circular mask area are then controlled by the phototherapy operation control module 216 (light-emitting control module) to perform the phototherapy operation.

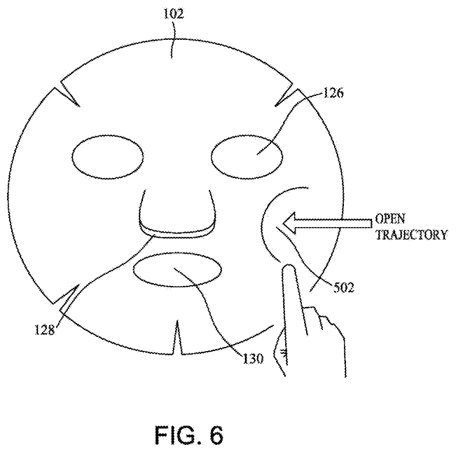

Referring to FIG. 6, an open trajectory touch selection on the mask body 100 is illustrated. The user's finger 506 is shown tracing an open trajectory 502 across the touch-sensitive layer 102. The open trajectory 502 may comprise a C-shaped, linear, or other non-closed path drawn by the user's finger 506 on the mask surface. When the touch operation type determination module 212 detects that the touch trajectory corresponds to the open trajectory 502, the touch operation type is identified as a trajectory selection type. The mask area determination module 214 determines a strip or band region along the open trajectory 502 path, with the touch width defining the width of the strip, as the mask area for phototherapy treatment.

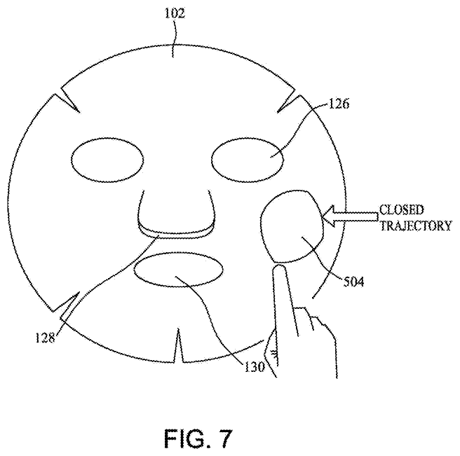

Referring to FIG. 7, a closed trajectory touch selection on the mask body 100 is illustrated. The user's finger 506 is shown tracing a closed trajectory 504 on the touch-sensitive layer 102, forming an enclosed loop on the mask surface. When the touch operation type determination module 212 detects that the touch trajectory forms the closed trajectory 504 where the end point meets or approaches the start point, the touch operation type is identified as a region selection type. The mask area determination module 214 determines the region enclosed by the closed trajectory 504 as the mask area for phototherapy treatment, with the boundary of the closed trajectory 504 having a thickness equal to the touch width.

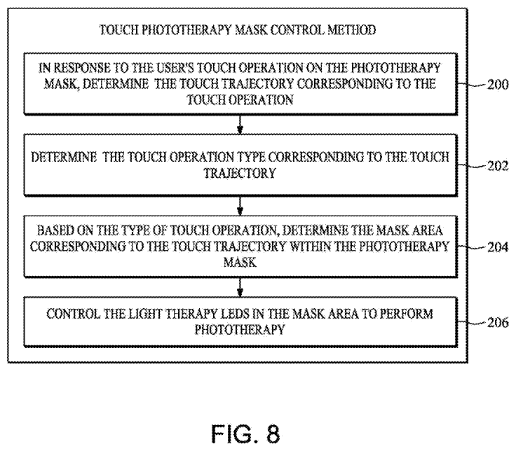

Referring to FIG. 8, a flowchart of a touch control method for the phototherapy device system is illustrated. The method begins at step 200, where the touch trajectory determination module 210 determines a touch trajectory corresponding to a detected touch operation on the touch-sensitive layer 102. The method proceeds to step 202, where the touch operation type determination module 212 determines the touch operation type corresponding to the touch trajectory, classifying the gesture as a single-point selection type, a trajectory selection type, or a region selection type. The method continues to step 204, where the mask area determination module 214 determines the mask area corresponding to the touch trajectory based on the determined touch operation type. The method concludes at step 206, where the phototherapy operation control module 216 (light-emitting control module) controls the LED beads 108 in the determined mask area to perform a phototherapy operation according to a target phototherapy method.

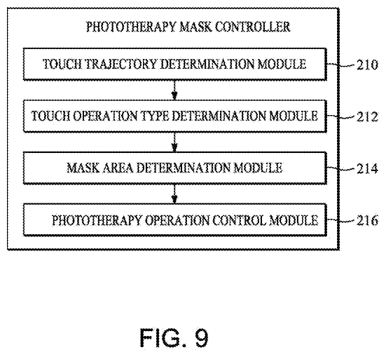

Referring to FIG. 9, a block diagram of a controller 112 of the phototherapy device system is illustrated. The controller 112 comprises a touch trajectory determination module 210 configured to determine the touch trajectory corresponding to the detected touch operation. The controller 112 further comprises a touch operation type determination module 212 configured to determine the touch operation type corresponding to the touch trajectory. The controller 112 further comprises a mask area determination module 214 configured to determine the mask area corresponding to the touch trajectory based on the touch operation type. The controller 112 further comprises a phototherapy operation control module 216 (light-emitting control module) configured to control the LED beads 108 in the determined mask area to perform phototherapy operations. The modules (210, 212, 214, and 216) are implemented as software modules executed by the processor 120 or as dedicated hardware circuitry within the controller 112.

In some cases, the touch-sensitive layer may comprise resistive sensors that detect touch operations through mechanical contact. The resistive sensors include two conductive layers separated by a small gap, where pressure applied to the outer layer causes contact between the two conductive layers at the touch location. The processor measures resistance values across the resistive sensor array to determine the location of the touch contact.

In some cases, the touch-sensitive layer may comprise pressure-sensitive sensors configured to detect both touch location and touch force. The pressure-sensitive sensors provide force measurements that are used to determine touch width or selection intensity. The pressure-sensitive sensors enable force-based scaling of selection regions, where greater touch force corresponds to larger selection areas.

The touch-sensitive layer is configured to detect a spatial location of a touch input and activate a corresponding subset of the plurality of light-emitting elements to emit light according to a phototherapy parameter. The touch-sensitive layer provides coordinate data indicating where on the mask body surface the user has made contact. The processor maps the detected spatial location to corresponding light-emitting elements positioned beneath the touch location. Upon determining the corresponding light-emitting elements, the processor controls the corresponding light-emitting elements to emit therapeutic light according to the selected phototherapy parameter.

The phototherapy device system interprets touch duration as an indicator of desired treatment intensity. When a user performs a long press by holding a finger in place on the touch-sensitive layer 102 for an extended period, such as two seconds or more, the processor 120 interprets this as a request for higher intensity treatment in that area. Conversely, a quick tap indicates that standard or lower intensity treatment is desired. The processor 120 measures the duration of each touch event and adjusts the assigned light intensity for the corresponding LED beads 108 in the treatment zone accordingly. This touch-duration-based intensity control provides an intuitive mechanism for users to indicate treatment priority without requiring separate intensity adjustment controls.

The touch-sensitive layer supports detection of multiple simultaneous touch points for multi-touch gesture recognition. The touch-sensitive layer tracks the movement of touch points across the surface over time to form touch trajectories. The touch-sensitive layer distinguishes between different types of touch contacts based on contact area, contact duration, or contact pressure characteristics. The touch-sensitive layer rejects unintentional contacts, such as palm resting on the surface, by analyzing contact characteristics and identifying contacts that do not match intentional gesture patterns.

Referring to FIG. 3, the LED beads 108 are arranged on the flexible light panel 106 in a distributed pattern corresponding to various treatment regions of the face. The LED beads 108 are organized into individually controllable clusters or zones, enabling selective activation of specific facial regions for targeted phototherapy treatment. The LED beads 108 comprise surface mount technology (SMT) LEDs, through-hole type LEDs, or high-power LEDs capable of emitting therapeutic wavelengths. The LED beads 108 include LEDs of multiple wavelengths distributed across the flexible light panel 106, allowing different treatment zones to emit different colors of light simultaneously or sequentially. The flexible light panel 106 comprises a flexible printed circuit board (PCB) or flexible circuit substrate that maintains electrical connections to the LED beads 108 while conforming to the curved contours of the mask body 100. The LED beads 108 are spaced at regular intervals across the flexible light panel 106 to provide uniform light coverage within each treatment zone. In some cases, the density of LED beads 108 may vary across different regions of the flexible light panel 106, with higher density in regions corresponding to areas requiring more intensive treatment.

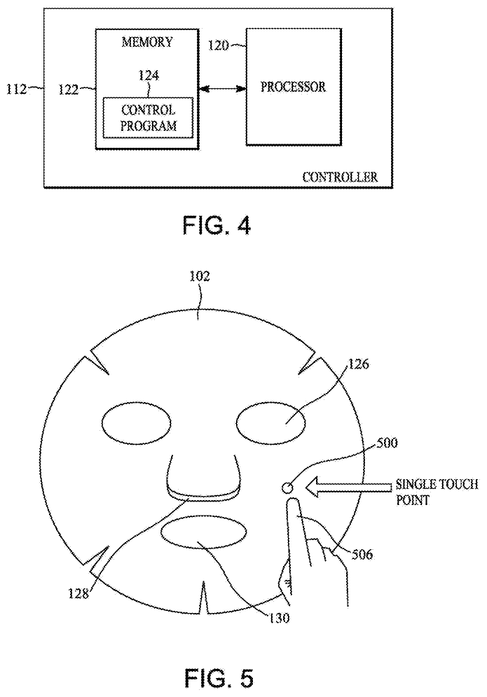

Referring to FIG. 4, the controller 112 comprises a processor 120 and a memory 122 storing a control program 124. The processor 120 executes the control program 124 to perform touch trajectory analysis, touch operation type classification, mask area determination, and LED control functions. The processor 120 comprises a central processing unit (CPU), microcontroller, digital signal processor (DSP), application-specific integrated circuit (ASIC), field-programmable gate array (FPGA), or other suitable processing device. The memory 122 comprises random access memory (RAM), read-only memory (ROM), flash memory, or other computer-readable storage media. The control program 124 includes software modules for processing touch input data, analyzing facial images, executing skin blemish recognition algorithms, and generating control signals for the LED beads 108. Referring to FIGS. 1 and 3, the controller 112 is housed in a separate enclosure connected to the mask body 100 via a connecting cable 114. The connecting cable 114 transmits touch coordinate data from the touch-sensitive layer 102 to the controller 112 and carries control signals and power from the controller 112 to the LED beads 108. A switch button 116 is provided on the controller 112 for powering on the device or initiating operational modes. A protective shell 132 is positioned at the junction where the connecting cable 114 attaches to the mask body 100, providing structural support for the connection interface.

In some cases, the mask body 100 may include a built-in microcontroller or ASIC realizing the processor 120, the memory 122, and a battery, eliminating the need for the external controller 112. The processor 120 and memory 122 may be embedded near the protective shell 132 or along the periphery of the flexible light panel 106. The touch-sensitive layer 102 signals may feed directly into the on-board processor 120. The switch button 116 may be relocated onto the mask body 100 itself, near the connecting positions 118, to allow on-mask activation or mode switching. Power may be supplied by a small rechargeable battery within the mask body 100 or via a detachable power module. Communication of the battery level or selected mode may be provided by indicator LEDs on the mask body 100 or by a wireless link to a smartphone.

The phototherapy device system includes a touch trajectory determination module 210 configured to determine a touch trajectory corresponding to a touch operation. The trajectory determination module is implemented as a software module executed by the processor or as dedicated circuitry within the controller. The trajectory determination module receives raw touch coordinate data from the touch-sensitive layer and processes the raw touch coordinate data to form a coherent touch trajectory representing the path traced by the user's touch input.

Referring to FIGS. 5 to 8, at method step 200, the processor is configured to determine a touch trajectory corresponding to the touch operation. When a user performs a touch operation on the phototherapy device, the processor continuously detects and records the coordinates of each touch point in chronological order. The processor samples touch coordinates at a predetermined sampling rate to capture the movement of the user's finger, stylus, or other touch device across the touch-sensitive surface. The sampling rate is selected to provide sufficient temporal resolution to accurately capture the shape and characteristics of the touch trajectory.

The touch trajectory is formed by ordering the recorded coordinate sequences according to the time at which each coordinate was detected. As the user moves a finger or stylus across the touch-sensitive surface, the trajectory determination module appends each newly detected coordinate to the existing sequence of coordinates. The resulting touch trajectory represents a continuous path from the initial touch point to the final touch point, capturing the complete movement pattern of the touch operation.

The trajectory determination module stores the recorded coordinate sequences in memory for subsequent processing by other modules of the phototherapy device system. The stored coordinate sequences include both spatial information indicating the position of each touch point on the touch-sensitive surface and temporal information indicating the time at which each touch point was detected. The temporal information is used to determine the speed and direction of movement along the touch trajectory.

The trajectory determination module processes touch operations performed with various touch instruments, including fingers, styluses, or other touch devices. When the user touches the mask surface with a finger, the trajectory determination module detects the contact location and tracks the movement of the finger across the surface. When the user touches the mask surface with a stylus, the trajectory determination module similarly detects the contact location and tracks the movement of the stylus tip. The trajectory determination module distinguishes between different types of touch instruments based on contact characteristics such as contact area or contact pressure.

The trajectory determination module filters the raw touch coordinate data to remove noise or spurious touch detections. The filtering includes smoothing operations that reduce jitter in the detected coordinates while preserving the overall shape of the touch trajectory. The filtering also includes rejection of touch points that fall outside expected ranges or that exhibit characteristics inconsistent with intentional touch operations.

The trajectory determination module detects when a touch operation begins and when the touch operation ends. The beginning of a touch operation is detected when the touch-sensitive layer first registers contact from the user's finger or stylus. The end of a touch operation is detected when the user lifts the finger or stylus from the touch-sensitive surface. The trajectory determination module uses the detected beginning and end points to define the boundaries of each discrete touch trajectory.

The trajectory determination module supports the detection of multiple concurrent touch trajectories when the user performs multi-touch operations. Each concurrent touch trajectory is tracked independently, with separate coordinate sequences maintained for each touch point. The trajectory determination module associates each detected touch point with the appropriate trajectory based on spatial proximity and temporal continuity of the touch coordinates.

Referring to FIGS. 5-8, at method step 202, the phototherapy device system includes a touch operation type determination module 212 configured to determine a touch operation type corresponding to the touch trajectory. The touch operation type determination module 212 examines the shape and characteristics of the touch trajectory to classify the gesture into one of several categories. The touch operation type includes a single-point selection type when the touch trajectory corresponds to a single touch point 500, as illustrated in FIG. 5. The touch operation type includes a trajectory selection type when the touch trajectory corresponds to an open trajectory 502, as illustrated in FIG. 6. The touch operation type includes a region selection type when the touch trajectory corresponds to a closed trajectory 504, as illustrated in FIG. 7. The touch operation type determination module 212 analyzes whether the end point of the touch trajectory meets or approaches the start point to distinguish between open and closed trajectories. The classification of the touch operation type is performed in real time as the user completes the touch gesture on the touch-sensitive layer 102.

Referring to FIGS. 5-8, at method step 204, the phototherapy device system includes a mask area determination module 214 configured to determine a mask area corresponding to the touch trajectory based on the touch operation type. When the touch operation type is the single-point selection type, the mask area determination module 214 determines a circular region centered on the single touch point as the mask area, with a preset touch width serving as the diameter of the circular region. When the touch operation type is the trajectory selection type, the mask area determination module 214 determines a strip or band region along the open trajectory path as the mask area, with the touch width defining the width of the strip on either side of the trajectory path. When the touch operation type is the region selection type, the mask area determination module 214 determines the region enclosed by the closed trajectory as the mask area, with the boundary of the closed trajectory having a thickness equal to the touch width. The mask area determination module 214 maps the determined mask area to corresponding LED beads 108 on the flexible light panel 106 for subsequent phototherapy activation.

The touch-sensing module of the phototherapy device system includes a force sensor configured to detect a touch force corresponding to the touch operation. The touch width is positively correlated with the touch force, such that a greater touch force corresponds to a greater touch width and a smaller touch force corresponds to a smaller touch width. A reference width and a corresponding reference force are preset in the memory 122, and the ratio of the detected touch force to the reference force is equal to the ratio of the resulting touch width to the reference width. In some cases, doubling the touch force results in doubling the selection width. This force-width mapping allows the user to adjust the size of the selected region naturally by pressing harder or softer on the touch-sensitive layer 102. The processor 120 receives force signals from the force sensor and calculates the corresponding touch width using stored calibration data and force-to-width mapping functions.

The phototherapy device system supports user-specific and region-specific touch width personalization. Different users are associated with different datum widths, such that the system performs personalized adaptation according to individual needs. When a user performs a touch operation on the phototherapy device, the user's identity information is determined by fingerprint recognition or other authentication means, and the corresponding reference width is retrieved from a preset database based on the identified user. Different fingers of the same user correspond to different datum widths to increase operational flexibility. The index finger is designated as the reference finger with the original reference width; when the user uses the thumb, the reference width is increased; when the user uses the little finger, the datum width is reduced. Different regions of the phototherapy device correspond to different datum widths to enhance operational adaptability. For delicate regions such as the nose opening 128 and the corners of the eye openings 126, the reference width is reduced; for broad regions such as the cheeks and the forehead, the reference width is increased.

The phototherapy device system includes a fingerprint sensor configured to detect fingerprint recognition information corresponding to the touch operation. Different finger types of the user correspond to different phototherapy methods, and the processor 120 is configured to identify a finger performing the touch operation based on the fingerprint recognition information and select a target phototherapy method corresponding to the identified finger. In some cases, when the user uses an index finger to perform a touch operation, the LED beads 108 in the corresponding mask area are controlled to perform phototherapy in a red light mode. When the user uses a thumb to perform the touch operation, the LED beads 108 in the corresponding mask area are controlled to perform phototherapy in a blue light mode. When the user uses a little finger to perform the touch operation, the LED beads 108 in the corresponding mask area are controlled to perform phototherapy in an infrared light mode. When the user uses a middle finger to perform the touch operation, the LED beads 108 in the corresponding mask area are controlled to perform phototherapy in a near-infrared light mode or another designated phototherapy mode. The fingerprint sensor is implemented using capacitive fingerprint sensing, optical fingerprint sensing, or ultrasonic fingerprint sensing technologies.

The phototherapy device system supports user-selected phototherapy mode and target method determination. A default phototherapy mode is preset in the memory 122, such as red light phototherapy, blue light phototherapy, infrared light phototherapy, or other phototherapy modes. After the mask area corresponding to the touch trajectory has been determined, the processor 120 controls the LED beads 108 disposed in the selected mask area to perform phototherapy according to the preset default phototherapy mode. In some cases, a target phototherapy mode corresponding to the user's touch operation is determined in response to the detected touch operation on the phototherapy device. The user selects the phototherapy mode via the touch interface on the touch-sensitive layer 102, via a connected mobile application, or via other user interface mechanisms associated with the phototherapy device.

The phototherapy device system stores a plurality of predefined phototherapy modes in the memory 122, each predefined phototherapy mode comprising a set of phototherapy parameters optimized for a specific skin condition or treatment goal. The predefined phototherapy modes include, for example, an acne treatment mode, an anti-aging mode, a skin brightening mode, a wound healing mode, and a relaxation mode. Each predefined phototherapy mode specifies the wavelength, intensity, duration, duty cycle, and pulsation parameters for the LED beads 108. The user selects a predefined phototherapy mode via the touch interface on the touch-sensitive layer 102, via the companion mobile application, or via the switch button 116 on the controller 112. Upon selection of a predefined phototherapy mode, the processor 120 automatically configures the LED beads 108 in the determined mask area to emit light according to the parameters of the selected predefined mode.

The phototherapy device system supports stylus-based color phototherapy selection. When performing a touch operation on the phototherapy device using a stylus, an end portion of the stylus is configured with different colored light sources that correspond to different phototherapy modes. When the user uses the stylus to perform a touch operation on the phototherapy device and a red light source of the stylus is activated, the LED beads 108 in the corresponding mask area are controlled to perform phototherapy in a red light mode. When a blue light source of the stylus is activated, the LED beads 108 in the corresponding mask area are controlled to perform phototherapy in a blue light mode. When an infrared light source of the stylus is activated, the LED beads 108 in the corresponding mask area are controlled to perform phototherapy in an infrared light mode. The touch-sensitive layer 102 detects the color of the stylus tip at the point of contact, and the processor 120 switches to the matching phototherapy mode.

The phototherapy device system supports mask area scaling and adjustment. The processor 120 is configured to scale and adjust the mask area in response to a mask area adjustment operation by the user to obtain an adjusted mask area. The user applies touch gestures to refine the selected mask area, such as tapping and dragging to enlarge or shrink the selected area, or drawing a new loop to redefine the boundary. The processor 120 scales or reshapes the mask area accordingly until the user is satisfied with the selection. The adjusted mask area is then used for phototherapy, with the LED beads 108 in the adjusted mask area controlled to perform the phototherapy operation according to the selected phototherapy mode.

The phototherapy device system supports undo and redo functionality for selection gestures. If a user makes an unintended selection gesture or wishes to revert to a previous selection configuration, the user performs a specific undo gesture, such as a three-finger swipe in a first direction or a shake gesture detected by an accelerometer in the mask body 100 or a connected device. The processor 120 maintains a history of recent selection states in the memory 122 and restores the previous selection configuration in response to the undo gesture. A redo gesture, such as a three-finger swipe in an opposite direction, restores a selection that was previously undone. This undo and redo functionality reduces user frustration and enables more confident experimentation with different treatment zone configurations.

The phototherapy device system supports multi-finger and gesture-based touch input for enhanced user interaction. The touch-sensitive layer 102 is configured to detect multi-touch gestures, and the processor 120 is configured to interpret the multi-touch gestures to manipulate the selection of treatment zones. A pinch gesture, where fingers move closer together, is interpreted as a command to reduce the size of a selected treatment area, while a spread gesture, where fingers move apart, is interpreted as a command to enlarge the selected treatment area. A two-finger rotation gesture is interpreted as a command to rotate the orientation of an elongated selection boundary. Multi-finger swipes are used to scroll through preset treatment profiles or cycle through light wavelength modes. The processor 120 distinguishes between single-finger gestures for zone selection and multi-finger gestures for zone manipulation, enabling intuitive and precise control over treatment area configuration.

Referring to FIG. 8, the phototherapy device system includes a phototherapy operation control module 216 (light-emitting control module) configured to control the LED beads 108 in the determined mask area to perform phototherapy operations. The phototherapy operation control module 216 (light-emitting control module) energizes only the LED beads 108 within the selected region on the flexible light panel 106, causing them to emit therapeutic light according to the target phototherapy method. LED beads 108 outside the selected region remain off, conserving power and avoiding unnecessary exposure to areas not requiring treatment. The phototherapy device system supports treatment of different regions sequentially or simultaneously with different phototherapy modes, as the controller 112 handles each selected area independently. The processor 120 generates control signals to activate the corresponding LED beads 108 at designated intensities and for required time intervals according to the determined phototherapy regimen.

The phototherapy device system provides display and visual feedback of the selected region to the user. The selected LED beads 108 briefly illuminate as a preview before treatment begins, allowing the user to verify the selected mask area. An overlay is shown on a connected application interface, displaying the selected region on a representation of the user's face. Indicator lights on the mask body 100 display the determined mask area to the user. The visual feedback is provided via a human-machine interface (HMI) on a stylus, mobile phone, tablet, computer, or other connected device. The user reviews the highlighted selection and confirms or modifies the mask area before initiating phototherapy treatment.

The phototherapy device system displays the determined phototherapy parameter to the user before or during treatment. The phototherapy parameter information is displayed on the companion mobile application, on a display integrated into the controller 112, or via indicator lights on the mask body 100. The displayed information includes the selected wavelength or light color, the treatment intensity level, the treatment duration, and the duty cycle. The user reviews the displayed phototherapy parameters and confirms or modifies them before treatment begins. During treatment, the phototherapy device system continues to display the active phototherapy parameters, remaining treatment time, and any parameter adjustments made by the system in response to real-time monitoring data.

The phototherapy device system provides gesture-based visual feedback scaling, where the mask body 100 displays light bars or patterns that scale in real time to show the user the size and extent of their selection as they perform gestures. When the user performs a pinch gesture to adjust the size of a selected area, indicator lights on the mask body 100 form a light bar whose length increases or decreases correspondingly to visually represent the changing selection size. For zoom-out gestures where fingers move closer together, the light bar length shortens accordingly, and for zoom-in gestures where fingers move apart, the light bar length increases accordingly. The light bar or pattern is displayed using a subset of the LED beads 108 that are not currently designated for treatment or using dedicated indicator lights positioned around the periphery of the mask body 100. The visual feedback also indicates the current touch force level through brightness variations in the displayed pattern.

The support layer 104 and the touch-sensitive layer 102 are transparent or semi-transparent, allowing active LED beads 108 to glow through the flexible light panel 106 so that the user can perceive which regions are being treated. When LED beads 108 corresponding to a detected blemish area or user-selected treatment zone are active, they illuminate the mask body 100 surface at that location. The user then double-taps that glowing spot on the touch-sensitive layer 102 either with a finger or a stylus to toggle phototherapy for that region. The touch-sensitive layer 102 maps the tap coordinates to the underlying LED beads 108, so a double-tap on a lit region starts or stops treatment in that zone. This feature provides an intuitive interface where the user simply touches the area of the mask body 100 where LED beads 108 are glowing to control the phototherapy, making it clear which areas are active and allowing direct touch control over the illuminated LED clusters.

In some embodiments, all layers of the mask body 100 are constructed from transparent or semi-transparent materials, such that the entire mask body 100 is optically transparent and the user or an observer can view the user's skin through the mask body 100 during treatment. The touch-sensitive layer 102 comprises a transparent capacitive film, the support layer 104 comprises a transparent or optically clear polymeric material, the flexible light panel 106 comprises a transparent flexible substrate carrying the LED beads 108, and the inner translucent bottom layer 110 comprises a transparent thermoplastic polyurethane (TPU) or polyurethane (PU) material. This fully transparent construction allows the user or a clinician to visually observe the skin surface through the mask body 100 during treatment, enabling real-time visual assessment of skin response to phototherapy without removing the mask body 100. The transparent construction also facilitates alignment of the mask body 100 with specific facial features or blemish regions during donning, and allows the imaging device to capture images of the skin through the mask body 100 during treatment for real-time monitoring and adaptive treatment adjustment.

The phototherapy device system supports confirmation and cancellation gestures for treatment zone selections. A double-tap gesture performed inside a selected treatment area is interpreted as confirmation of the selection and initiates phototherapy treatment in that area. A double-tap gesture performed outside any selected treatment area is interpreted as cancellation of all current selections. A specific gesture, such as drawing an X pattern across the mask surface, clears all current selections and resets the mask to an unselected state. A triple-tap gesture toggles between a preview mode, where selected areas are highlighted but not treated, and an active treatment mode where phototherapy is delivered to selected areas. These distinct confirmation and cancellation gestures reduce accidental treatment initiation and provide clear user control over the treatment process.

In a touch-based operational sequence, upon powering on via the switch button 116 or a touch gesture, the phototherapy device system enters a standby mode or applies a predefined default phototherapy pattern across all LED beads 108 if no touch input is detected. A low-power mode maintains sensor readiness on the touch-sensitive layer 102. On single tap detection by the user's finger 506 or stylus at one location, the system immediately designates a circular region around that single touch point 500 with a diameter equal to the current touch width as the active mask area, and the phototherapy operation control module 216 (light-emitting control module) activates the LED beads 108 within that region. If the user drags a finger or stylus creating an open trajectory 502, the trajectory selection type is identified, and the mask area corresponding to the region swept by the drag path is determined. On performing a closed-loop gesture forming a closed trajectory 504, the enclosed region is identified as an area-selection type. The user then double-taps inside/outside the enclosed area to confirm or cancel. Throughout these interactions, the detected touch force is continuously measured by the touch-sensitive layer 102, and the touch width is scaled proportionally to the force. Once the user confirms the selection, the phototherapy operation control module 216 (light-emitting control module) energizes the selected LED beads 108 in the chosen mode. The LED beads 108 remain active for the programmed duration or until the user signals termination by a double-tap on the region or pressing the switch button 116.

The phototherapy device system supports a standby state for the light-emitting elements before initiating phototherapy treatment. When the user selects a treatment zone via touch gestures on the touch-sensitive layer 102 or via AI-based detection, the light-emitting control module places the plurality of LED beads 108 corresponding to the user-selected region in a standby state. In the standby state, the LED beads 108 are powered but do not emit therapeutic light at full intensity, or emit a low-intensity preview illumination to indicate the selected treatment zone to the user. The standby state allows the user to verify and confirm the selected region before the LED beads 108 transition to full therapeutic emission according to the determined phototherapy parameter.

In a practical use scenario combining both touch and AI modes, the user fits the mask body 100 onto their face, aligning the eye openings 126 and adjusting the straps through the connecting positions 118. The mask body 100 conforms closely to the face due to the strap tension and the flexibility of the mask layers. The user presses the switch button 116 or gives a command via a connected external device to start the system. The processor 120 initializes, and the imaging device captures the user's facial image for AI-based blemish detection. The skin blemish recognition model identifies blemish regions and proposes treatment zones, which are displayed as illuminated LED beads 108 on the mask body 100 for user review. The user confirms the AI-detected zones via a double-tap on the touch-sensitive layer 102, or modifies the zones by drawing additional touch trajectories to add or remove treatment areas. The user also selects additional treatment zones manually using touch gestures such as single-point taps, open trajectories 502, or closed trajectories 504 on the touch-sensitive layer 102. Once all treatment zones are confirmed, the phototherapy operation control module 216 activates the LED beads 108 in each zone according to the determined phototherapy regimens. During treatment, the user adjusts zones on-the-fly by double-tapping to toggle individual regions, or presses the switch button 116 to end the session. Treatment session data is stored in the memory 122 and optionally uploaded to the cloud for longitudinal tracking.

The phototherapy device system supports contactless hover selection and proximity detection for improved hygiene. The phototherapy device includes proximity sensors configured to detect a hover gesture by the user at a distance from the touch-sensitive layer 102 without physical contact. The proximity sensors detect the position of a user's finger or stylus when held within a predetermined distance from the touch-sensitive layer 102, such as within 1 to 3 centimeters of the surface. The processor 120 interprets hover gestures like touch gestures, recognizing single-point hover positions, open hover trajectories, and closed hover loops to determine corresponding mask areas for phototherapy. The proximity sensors are implemented using infrared proximity sensors, capacitive proximity sensors, or time-of-flight sensors. This contactless interaction mode enhances both hygiene and convenience, particularly for users with sensitive skin conditions or in environments where minimizing surface contact is desirable.

The phototherapy device system includes a haptic feedback mechanism configured to provide vibration feedback on the inner translucent bottom layer 110 corresponding to a selected treatment pattern when the user touches the touch-sensitive layer 102. The haptic feedback mechanism is implemented using eccentric rotating mass motors, linear resonant actuators, or piezoelectric actuators. The haptic actuators are distributed across the inner translucent bottom layer 110 at positions corresponding to different treatment zones, such that when the user touches a location on the touch-sensitive layer 102, the corresponding location on the inner translucent bottom layer 110 provides tactile confirmation through vibration. The vibration patterns vary in intensity, frequency, or duration to convey different types of information to the user, such as confirming selection, indicating treatment progress, or alerting the user to reposition the mask body 100.

The phototherapy device system incorporates palm rejection and idle object detection functionality to distinguish intentional selection gestures from unintentional contact. When the touch-sensitive layer 102 detects contact characteristics consistent with an idle object, such as a palm resting on the mask surface rather than an intentional finger or stylus gesture, the processor 120 determines that the contact is unintentional and refrains from interpreting the contact as a treatment zone selection. The processor 120 identifies idle objects based on contact area size, contact duration, lack of movement, or other distinguishing characteristics. When an idle object is detected, the phototherapy device system provides feedback to alert the user, such as causing the LED beads 108 to flash or blink in a distinctive pattern, or providing haptic feedback through the haptic feedback mechanism. The sensitivity of the idle object detection is adjustable based on user preferences stored in the memory 122.

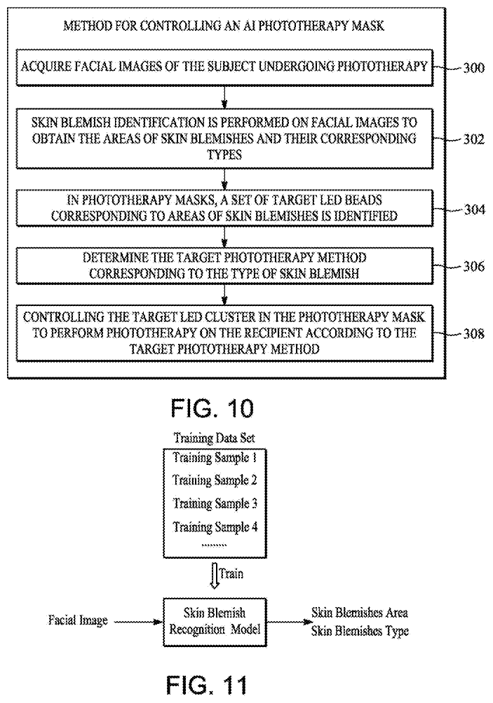

Referring to FIG. 10, in AI-based automatic detection mode, the phototherapy device system acquires facial images of the user at method step 300. The phototherapy device system includes an imaging device configured to capture one or more facial images of the subject to be treated. The imaging device comprises a camera integrated into the mask body 100 or connected externally to the controller 112. The acquired images include a visible light image captured under normal illumination and, optionally, an ultraviolet (UV) image captured under UV illumination for deeper skin analysis. The facial images are time-stamped and stored in the memory 122 for the current session. A facial image acquisition module 320 is configured to control the imaging device and manage the image capture process.

Referring to FIGS. 10 and 11, at method step 302, the acquired facial images are processed using a pretrained skin blemish recognition model to detect and classify skin blemishes. A skin blemish identification module 322 processes the captured images using the pretrained artificial intelligence model to detect blemish regions and classify their types and severity. The skin blemish recognition model segments the facial image into regions and classifies each detected blemish, outputting for each blemish object its spatial region, an associated blemish type label, and a severity score. The skin blemish recognition model is trained using a training dataset comprising multiple training samples, each training sample including one or more sample facial images and corresponding annotation data indicating the blemish regions and blemish types. During training, sample facial images are input to an initial AI model, and the outputs are compared to the annotation data to compute a training loss according to a predefined loss function. Model parameters are iteratively updated by backpropagation to reduce the loss until preset training termination conditions are satisfied, such as reaching a predetermined number of training iterations or achieving model convergence. The skin blemish recognition model is trained on both visible light images and UV images to detect different types of blemishes.

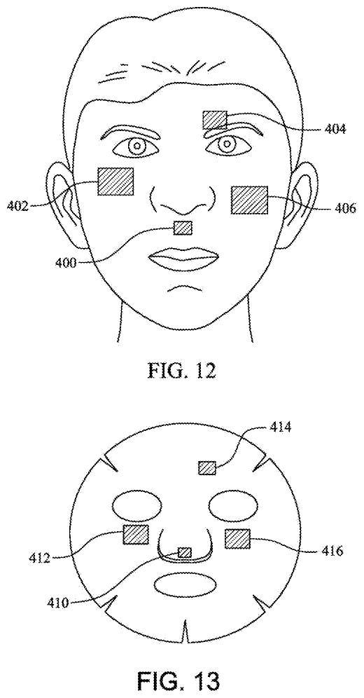

Referring to FIG. 12, the skin blemish recognition model outputs, for each detected blemish, its spatial region, blemish type, severity level, and a confidence score. The confidence score is expressed as a percentage value ranging from 0% to 100%, or as a decimal value ranging from 0 to 1, indicating the model's certainty in the detection and classification. The processor 120 uses the confidence scores to filter detections, excluding detections with confidence scores below a predetermined threshold from the treatment plan. Detections with confidence scores in an intermediate range are flagged for user review, allowing the user to confirm or reject uncertain detections before treatment proceeds. High-confidence detections are automatically included in the treatment plan without requiring user confirmation. The skin blemish recognition model also measures and outputs the precise size of each detected skin blemish region, expressed in pixels, millimeters, square millimeters, or as a percentage of the total facial area.

The severity level for each detected skin blemish region is determined based on one or more characteristics of the blemish, including the size of the blemish region, the intensity or color depth of the blemish relative to surrounding skin, the density of blemish features within the detected region, or the spatial distribution of the blemish across the facial area. For example, a blemish region with a high density of acne lesions concentrated in a small area is assigned a higher severity level than a region with sparse, widely distributed lesions of similar count. The spatial distribution indicates whether blemishes are clustered in a localized area or spread across multiple facial zones, which influences the treatment regimen by adjusting the number of target LED sets activated and the intensity applied to each zone.

Referring to FIG. 12, the blemish types detected by the skin blemish recognition model include acne, eczema, freckles, hyperpigmentation, age spots, scars, rosacea, and other dermatological condition categories. As shown in FIG. 12, distinct skin blemish regions are detected on the face, including a first skin blemish region 400, a second skin blemish region 402, a third skin blemish region 404, and a fourth skin blemish region 406. It will be appreciated that the number, location, size, and types of blemishes shown are exemplary and do not impose limitations on practical application; actual recognition results vary with individual subjects and their specific dermatological conditions.

Referring to FIGS. 12 and 13, at method step 304, each detected facial blemish region is mapped to a corresponding set of target LED beads 108 on the mask body 100. An LED bead collection determination module 324 maps each identified facial blemish region to the corresponding set of LED beads 108 by utilizing a stored face-to-mask coordinate mapping. As shown in FIG. 13, the first skin blemish region 400 is mapped to a first target LED set 410, the second skin blemish region 402 is mapped to a second target LED set 412, the third skin blemish region 404 is mapped to a third target LED set 414, and the fourth skin blemish region 406 is mapped to a fourth target LED set 416. The calibration process includes factory pre-calibration with a standard facial model, user-specific calibration during initial setup, or session-specific calibration using captured images. Facial landmarks such as eye corners, nose bridge, mouth corners, and jawline points are detected in the facial image and are correlated with known positions on the mask body 100 to compute a face-to-mask coordinate transform matrix, which is stored in the memory 122.

The face-to-mask coordinate mapping employs a two-dimensional geometric transform for simpler implementations, including translation, rotation, and scaling operations to align the facial image coordinates with the mask LED grid coordinates. In some cases, a three-dimensional mesh-based calibration is employed for more precise mapping, utilizing depth sensing, structured light projection, or stereo imaging to capture the three-dimensional geometry of the user's face. The detected blemish coordinates are projected onto the 3D facial model and subsequently mapped onto the mask's LED grid, accounting for facial curvature, contours, and asymmetries. The phototherapy device system performs dynamic recalibration during use if the mask body 100 shifts or moves relative to the face. Fit sensors embedded in the mask body 100 detect changes in mask position, and the processor 120 triggers a recalibration routine to update the face-to-mask mapping accordingly. The processor 120 performs calibration verification before initiating treatment to ensure accurate mapping.

Referring to FIG. 10, at method step 306, a target phototherapy regimen is determined for each detected skin blemish region based on the corresponding blemish type and severity level. A phototherapy mode determination module 326 selects phototherapy regimen parameters, including light wavelength, intensity, and duration for each blemish based on its type and severity, using a predefined mapping between blemish types and phototherapy regimens. For example, a blemish of type B1 corresponds to red light phototherapy with treatment duration T1, a blemish of type B2 corresponds to blue light phototherapy with treatment duration T2, and a blemish of type B3 corresponds to infrared phototherapy with treatment duration T3. The phototherapy regimen is scaled based on the estimated severity level, such that more severe blemishes receive longer or more intensive treatment than mild blemishes. For a given blemish type, a mild severity level corresponds to a short treatment cycle, a moderate severity level corresponds to a moderate treatment cycle, and a severe severity level corresponds to a longer treatment cycle.

The phototherapy device system presents the detected skin blemish regions and corresponding blemish types to the user via a human-machine interface for verification. The user confirms the recognition results or modifies them, such as by reassigning the blemish type associated with a detected region or by adding a missed blemish region. Where modifications are provided, the processor 120 uses the modified blemish type information for subsequent processing and regimen selection. User corrections are saved in the user profile stored in the memory 122 for future model refinement and personalization.

The phototherapy device system performs skin tone analysis on the acquired facial image to obtain skin tone information of the subject. The extracted skin tone information is supplied to the skin blemish recognition model or is used to select a skin-tone-specific preprocessing routine so that blemish detection is accurate across different complexions. The training dataset used to train the skin blemish recognition model includes skin color annotations so that the trained model learns a mapping from image data and skin color information to accurate blemish detection and classification. This skin-tone-aware approach reduces bias and improves recognition accuracy across users with varying skin tones.

In some embodiments, the image acquisition stage incorporates correction for non-frontal capture angles to reduce recognition error introduced by varied shooting angles. The processor 120 determines the capture angle or pose associated with the captured facial image. When the facial image is acquired from a lateral or oblique viewpoint, the processor 120 performs geometric correction, such as frontalization, to transform the image to approximate a frontal facial view. Photometric normalization may also be applied to account for variations in lighting conditions across the captured image. Subsequent recognition and mapping operations operate on the corrected facial image to improve the accuracy of blemish detection and face-to-mask coordinate mapping.

The controller 112 may include a facial image correction module configured to determine the shooting angle or pose associated with a captured facial image and to perform image correction processing based on the determined shooting angle. The facial image correction module may estimate the face pose from the captured image using facial landmark detection and geometric analysis. Based on the estimated pose, the facial image correction module may apply a geometric transformation to produce a corrected frontal facial image, with or without photometric normalization, for use in subsequent recognition and mapping operations. The corrected frontal facial image ensures that the skin blemish recognition model receives a near-frontal facial image regardless of the original capture angle, reducing pose-related errors in blemish region detection.

The skin blemish identification module 322 may be configured to process the corrected facial image and, based on the corrected image, detect skin blemish regions and ascertain the corresponding skin blemish types. By operating on the corrected facial image rather than the original non-frontal image, the skin blemish identification module 322 may achieve improved detection accuracy and more reliable spatial localization of blemish regions. The corrected facial image may also improve the accuracy of the face-to-mask coordinate mapping performed by the LED bead collection determination module 324, as the geometric relationship between facial landmarks and mask coordinates is more consistent when computed from a frontal facial view.

The phototherapy device system maintains a user profile that stores past facial images, blemish annotations, and treatment parameters for each user. The processor 120 retrieves historical phototherapy records associated with the subject and compares the current facial images with facial images stored in the historical records to determine treatment progress. The comparison quantifies changes such as percentage reduction in blemish area or changes in severity level. The phototherapy mode determination module 326 adapts future regimens based on the determined treatment progress, such that stubborn lesions that have not improved under previous settings receive longer or different wavelength treatment, while resolved lesions receive reduced exposure.

Referring to FIG. 10, at method step 308, the phototherapy device system combines AI-based detection with touch-based user input in a hybrid operation mode. An AI phototherapy mask control module 328 issues control commands to activate the target LED beads 108 according to the determined phototherapy regimens. The LED beads 108 in the target regions illuminate as a preview, allowing the user to verify the AI-detected blemish regions before treatment begins. The user confirms the AI-detected regions via a double-tap gesture on the illuminated region on the touch-sensitive layer 102. The user also modifies the AI-detected regions using touch gestures, such as adding missed blemish regions by drawing on the touch-sensitive layer 102 or removing false detections by double-tapping outside the detected region. This hybrid approach combines the benefits of automatic AI-based analysis with user control over the treatment process.

The phototherapy device system supports wireless connectivity to external devices for enhanced functionality and user interaction. The mask body 100 connects wirelessly to an external device such as a smartphone, tablet, or computer via Bluetooth, Wi-Fi, or other wireless communication protocols. A companion mobile application installed on the external device implements touch trajectory logic and displays the selected treatment area on the device screen for user confirmation. The mobile application sends the final mask area and phototherapy mode back to the controller 112, which then activates the corresponding LED beads 108. In some cases, if no controller 112 is attached, the connecting cable 114 serves only as a power cable, or the connecting cable 114 is omitted entirely when the mask body 100 includes an integrated battery. The companion mobile application provides treatment scheduling features, including calendar integration, customizable reminders, and notification alerts for upcoming or missed sessions. A progress photo gallery feature allows the user to capture and store facial images at regular intervals, with side-by-side comparison views to visualize improvement over time. The application displays treatment history in graphical form, showing trends in treatment frequency, duration, and detected blemish counts.

The phototherapy device system supports pre-treatment symbol mapping using an external device such as a smart mirror or mobile phone. Before wearing the mask body 100, the user stands in front of the smart mirror or positions their face in view of the mobile phone camera and marks areas needing treatment using predefined symbols on the skin or via the device interface. The symbols include a minus sign to indicate one type of treatment, an O shape to indicate another type of treatment, and a zigzag pattern to indicate yet another type of treatment. The external device captures an image of the user's face with the marked symbols and transmits the image data to the controller 112 via Bluetooth or Wi-Fi. The controller 112 processes the captured image to identify the locations and types of symbols marked on the face. Pre-stored mappings between symbols and phototherapy modes are maintained in the memory 122, such that areas marked with a minus sign receive red light therapy, areas marked with an O shape receive blue light therapy, and areas marked with a zigzag pattern receive green light therapy. When the mask body 100 is subsequently worn, the designated areas receive the corresponding treatment without requiring additional touch input on the touch-sensitive layer 102.

The phototherapy device system supports serum detection and topical application adjustment for enhanced therapeutic efficacy. Mappings between different types of topical phototherapy application substances, such as serums, creams, or other treatment agents, and corresponding phototherapy modes are predefined and stored in the memory 122. After the mask area corresponding to the detected touch trajectory is determined, a sensor integrated into the inner translucent bottom layer 110 detects the type of phototherapy application substance present within the selected mask area. The processor 120 determines an appropriate target phototherapy mode according to the detected substance type and controls the LED beads 108 in the mask area to perform phototherapy according to the determined target mode. For example, if the detected phototherapy application substance in the mask area is of a first type, the LED beads 108 in the mask area are controlled to perform red light phototherapy; if the detected substance is of a second type, the LED beads 108 are controlled to perform blue light phototherapy; and if the detected substance is of a third type, the LED beads 108 are controlled to perform infrared phototherapy.

The phototherapy device system detects various types of indications associated with the user-selected region to determine the phototherapy parameter. The indications include therapeutic substances such as serums or creams applied to the skin, color markings drawn on the skin by the user to indicate desired treatment areas, symbols such as predefined shapes or characters marked on the skin or indicated via an external device, patterns such as geometric designs or coded markings, or other visually or sensor-detectable markers placed on or near the skin surface. The processor 120 detects the indications using the imaging device, sensors integrated into the inner translucent bottom layer 110, or data received from an external device. Based on the type of detected indication, the processor 120 determines the appropriate phototherapy parameter, such as selecting a specific wavelength, intensity, or duration corresponding to the detected indication.

The phototherapy device system includes a skin condition scanner for automatic treatment mapping. The skin condition scanner comprises a camera or sensor array incorporated into the mask body 100 or a connected external device, such as a smart mirror or mobile phone. The scanner captures facial images and analyzes skin conditions using image processing algorithms to detect and map various skin conditions, including acne, pigmentation, wrinkles, inflammation, and sebum levels. Based on the detected conditions, the processor 120 automatically generates treatment zone recommendations and suggests appropriate wavelengths for each identified area. For instance, areas identified as having acne are automatically assigned blue light therapy, while areas showing signs of aging or requiring regeneration are assigned red or near-infrared light therapy. The processor 120 overlays the detected skin condition map onto the mask area grid corresponding to the LED beads 108 on the flexible light panel 106, enabling the user to review and confirm the automatically suggested treatment zones before initiating phototherapy.

The phototherapy device system supports multi-region simultaneous treatment with independent parameters for each region. The user selects a first treatment zone using a first touch gesture on the touch-sensitive layer 102 and assigns a first wavelength, such as red light, to the first zone. The user then selects a second treatment zone using a second touch gesture and assigns a second wavelength, such as blue light, to the second zone. The processor 120 maintains separate treatment parameters for each selected zone and controls the corresponding LED beads 108 independently. For example, one cheek receives red light therapy for skin regeneration while the other cheek region simultaneously receives blue light therapy for acne treatment. Similarly, the forehead region receives high-intensity treatment while the chin region receives low-intensity treatment. This multi-zone capability enables comprehensive facial treatment protocols within a single treatment session, with each zone receiving optimized phototherapy parameters based on the specific skin condition in that region.

The phototherapy device system supports per-zone light intensity control, where different mask areas receive different light intensities simultaneously, in addition to different wavelengths. Each LED module or group of LED beads 108 is individually controllable for both wavelength selection and intensity level. Intensity control is implemented through pulse width modulation, current regulation, or other suitable dimming techniques. For example, a first mask area corresponding to a region with severe acne receives blue light at a higher intensity level, while a second mask area corresponding to a region with mild skin concerns receives the same wavelength at a lower intensity level. The light intensity for each zone is determined based on the detected skin condition severity, user preferences, or professional prescription. The touch force detected during zone selection also influences the assigned intensity, where a harder press during selection indicates a desire for higher intensity treatment in that area. The per-zone intensity control optimizes treatment efficacy while minimizing unnecessary light exposure to areas requiring less intensive treatment.

The phototherapy device system supports pulsed light delivery with a configurable duty cycle and pulse frequency. The LED beads 108 are activated and deactivated in rapid cycles rather than continuous illumination. The duty cycle is configurable to values such as 50%, 75%, or 90%, and the pulse frequency ranges from a few Hertz to several kilohertz. Pulsed light delivery provides benefits including reduced heat generation at the skin surface, improved light penetration into tissue layers, and optimized cellular response to phototherapy. The phototherapy regimen stored in the memory 122 specifies whether pulsed or continuous mode is to be used for each treatment zone. The controller 112 implements pulse width modulation (PWM) to control the duty cycle and timing of the LED beads 108. The processor 120 synchronizes pulsing across multiple LED clusters when treating adjacent zones to maintain consistent therapeutic delivery. User or clinician controls are provided to select between pulsed and continuous modes based on the specific treatment requirements.

The pulsation parameters are configured differently for different wavelengths, as optimal pulsation characteristics vary depending on the light wavelength and target tissue depth. For example, near-infrared light intended for deeper tissue penetration uses different pulsation parameters than blue light intended for surface-level treatment. The pulsation mode is selected automatically based on the treatment protocol or manually by the user, and the system provides visual or haptic feedback through the haptic feedback mechanism to indicate when the pulsation mode is active.

The phototherapy device system supports combination therapy protocols that apply multiple wavelengths sequentially or simultaneously to a treatment area. In sequential combination therapy, a first wavelength is applied for a specified duration, followed by a second wavelength. For example, blue light is applied first to target bacteria in an acne-affected region, followed by red light to promote healing and reduce inflammation. In simultaneous combination therapy, LED beads 108 of different wavelengths are activated concurrently within the same treatment zone to provide combined therapeutic effects. Predefined combination protocols are stored in the memory 122 for specific conditions such as inflammatory acne or post-inflammatory hyperpigmentation. The processor 120 executes the combination protocol by controlling the timing, intensity, and sequencing of different wavelength LED beads 108 according to the stored protocol parameters.

The phototherapy device system supports time-sequenced multi-wavelength treatment protocols where different wavelengths are automatically applied in sequence within a single treatment session. The processor 120 executes pre-programmed treatment sequences that transition between different light wavelengths at specified time intervals. For example, a treatment session begins with blue light in the 400-470 nm wavelength range for sterilization during a first time period such as the initial three minutes, followed by yellow light in the 580-595 nm wavelength range for disinfection during a second time period such as minutes three through six, and then red light in the 620-670 nm wavelength range for regeneration during a third time period such as minutes six through nine. The sequencing is customized based on the user's skin condition, age, or treatment goals. Different mask areas follow different sequences simultaneously. The time-sequenced protocols are stored in the memory 122 and are selected by the user or automatically recommended based on detected skin conditions.

The phototherapy device system includes fit sensors integrated into the inner translucent bottom layer 110 to monitor contact between the mask body 100 and the user's skin. The fit sensors comprise miniature pressure sensors, capacitance sensors, or proximity sensors positioned at various points across the inner translucent bottom layer 110. The fit sensors detect whether the mask body 100 is fully seated against the user's face by measuring pressure, capacitance changes, or distance at each sensor location. If a fit sensor detects that the mask body 100 is not fully seated in a particular region, as indicated by low pressure or increased distance, the processor 120 infers that a gap exists between the mask body 100 and the skin in that area. The controller 112 compensates for the detected gap by adjusting the phototherapy output, such as increasing intensity or duration for that zone, or by alerting the user to adjust the mask body 100. The fit sensors ensure consistent irradiation across the treatment area, even if the mask body 100 shifts or sits unevenly during the treatment session.

The phototherapy device system includes temperature sensors positioned on the inner translucent bottom layer 110 to monitor skin temperature during treatment. The temperature sensors comprise thermistors, thermocouples, infrared temperature sensors, or resistance temperature detectors positioned at various locations corresponding to different treatment zones. The processor 120 samples temperature readings from the temperature sensors at regular intervals during the treatment session and compares the readings against predefined safety thresholds stored in the memory 122. If the monitored temperature at any location exceeds a predetermined safety threshold, the processor 120 automatically reduces the intensity of the LED beads 108 in that region, pauses the treatment, or terminates the session entirely. The controller 112 provides an alert to the user via visual feedback on indicator lights, audible feedback through a speaker, or haptic feedback through the haptic feedback mechanism to indicate that a temperature threshold has been exceeded.

The phototherapy device system incorporates a thermal management system to dissipate heat generated by the LED beads 108 during operation. Passive cooling mechanisms include heat sinks, thermally conductive materials integrated into the support layer 104 or flexible light panel 106, and ventilation channels that allow airflow across the mask body 100 surface. In some cases, active cooling mechanisms are employed, such as micro-fans positioned at the periphery of the mask body 100 or thermoelectric cooling elements that actively transfer heat away from the LED beads 108. The thermal management system maintains a comfortable temperature at the skin interface during extended treatment sessions, preventing thermal discomfort or skin irritation. The processor 120 monitors temperature data from the temperature sensors and adjusts the thermal management system operation accordingly. In some cases, the system displays a temperature warning to the user or pauses treatment until the mask body 100 cools to a safe operating temperature.

The phototherapy device system includes adverse reaction detection and automatic safety shutoff features. The imaging device or a separate camera system captures images of the user's skin at periodic intervals during the treatment session, such as every 30 seconds or every minute. The controller 112 analyzes the captured images to detect signs of adverse reactions, including excessive redness, swelling, or irritation that indicate the skin is responding negatively to the phototherapy. If such signs are detected, the processor 120 automatically reduces the LED intensity, pauses the treatment, terminates the session, or alerts the user. An automatic shutoff mechanism terminates LED operation if the treatment duration exceeds a preset maximum threshold. A motion sensor or accelerometer integrated into the mask body 100 detects if the mask is removed during treatment, triggering an automatic pause or shutoff. Eye protection features include opaque regions or reduced LED density near the eye openings 126. The processor 120 also monitors LED current and voltage to detect malfunctions and disables affected LED clusters if anomalies are detected.

The phototherapy device system implements real-time skin monitoring and adaptive treatment that dynamically adjusts parameters based on skin response observed during the session. The processor 120 analyzes images captured by the imaging device during treatment to detect changes in skin appearance, such as increasing redness or blanching. Based on the detected changes, the processor 120 automatically modulates LED intensity, duty cycle, or wavelength mix to optimize therapeutic delivery while minimizing the risk of overtreatment or adverse reactions. This closed-loop control approach provides personalized treatment adaptation in real time. A post-treatment image is captured immediately after the session concludes and is compared with the pre-treatment image to assess immediate skin response. Before-and-after images are stored in the treatment history associated with the user profile in the memory 122 for longitudinal progress tracking. The monitoring sensitivity is adjustable, with higher sensitivity settings triggering safety responses at lower thresholds of detected skin changes.

The phototherapy device system performs contraindication checks and provides treatment guidance to ensure safe and appropriate use. Before initiating a treatment session, the processor 120 prompts the user to confirm the absence of contraindications such as photosensitizing medications, active skin infections, or recent cosmetic procedures that are adversely affected by phototherapy. The system maintains a database of known contraindications in the memory 122 and provides warnings or prevents treatment initiation if contraindications are indicated. Treatment guidance includes recommendations for treatment frequency, session duration, and post-treatment care based on the detected skin conditions and selected phototherapy regimens. The companion mobile application provides educational content regarding phototherapy, skin health, and proper device usage to support informed treatment decisions.

The phototherapy device system supports treatment scheduling and recommended protocols through a connected smart device application. The user configures a treatment schedule specifying preferred treatment times, treatment duration, treatment zones, and wavelength selections for each scheduled session. The smart device application generates reminder notifications at scheduled treatment times to prompt the user to initiate treatment. The system tracks treatment compliance by recording whether scheduled treatments were completed and generates compliance reports over time. The scheduling system suggests optimal treatment times based on the user's historical usage patterns or based on circadian rhythm considerations for skin treatment efficacy. Recommended protocols for specific skin conditions are provided based on clinical guidelines or derived from aggregated anonymized treatment outcome data.

The recommended treatment frequency varies depending on the blemish type and severity, for example, ranging from 3 to 5 treatment sessions per week for active acne to 2 to 3 sessions per week for maintenance therapy. Each treatment session has a recommended duration, which ranges from 5 to 30 minutes depending on the treatment area and light wavelength used. The system recommends a treatment course consisting of a specified number of sessions over a defined period, for example, 8 to 12 weeks, followed by a rest period before beginning a subsequent course. The system prevents or warns against excessive treatment by enforcing minimum intervals between sessions or maximum cumulative exposure limits within a defined time period.

The phototherapy device system includes automatic timing and safety shutoff features to prevent overexposure. The processor 120 implements countdown timers for each treatment zone that track elapsed treatment time and automatically deactivate the corresponding LED beads 108 after a preset treatment duration has been reached. The preset duration is configured by the user, recommended by the system based on detected skin conditions, or prescribed by a healthcare professional. The system provides audible alerts such as beeps, visual alerts such as flashing indicator lights, or haptic alerts such as vibration pulses through the haptic feedback mechanism to notify the user when treatment is nearing completion or has completed. The automatic shutoff feature prevents overexposure and enhances treatment safety, particularly for users who fall asleep during treatment or lose track of time.

The phototherapy device system supports multiple user profiles to accommodate use by different individuals, such as family members. Each user profile stored in the memory 122 includes personalized calibration data for face-to-mask coordinate mapping, treatment history including past facial images and detected blemish records, preferred settings such as default phototherapy modes and touch width preferences, and user-specific reference width values. Users switch between profiles via the touch interface on the touch-sensitive layer 102, via the companion mobile application, or via facial recognition performed by the imaging device. The system maintains data separation between profiles to preserve privacy and ensure that treatment recommendations are based on the correct user's history. Profile-based calibration enables accurate face-to-mask mapping for users with different facial geometries.

The phototherapy device system supports demographic and skin type personalization through user profile settings that customize default treatment parameters. When a user creates a profile, the user inputs personal information, including age group, gender, skin type, such as oily, dry, combination, or sensitive, and geographic location or climate conditions. The processor 120 uses this profile information to automatically adjust default treatment parameters and recommend appropriate phototherapy regimens. For example, users in younger age groups receive treatment protocols focused primarily on sterilization and acne treatment, while users in older age groups receive protocols that additionally include skin regeneration and anti-aging phases. Users with dry skin or in dry climate conditions receive protocols with reduced blue light exposure time to prevent excessive skin drying. Different skin types have different default intensity levels and wavelength preferences. These personalized defaults are overridden by manual user selection or professional prescription when desired.

The phototherapy device system supports cloud connectivity and data synchronization for enhanced functionality and remote management. The controller 112 includes a wireless communication module, such as Wi-Fi, Bluetooth, or cellular connectivity, that connects the device to a cloud platform. Treatment data, including captured facial images, detected blemish information, selected phototherapy regimens, and treatment outcomes, are uploaded to the cloud server with user permission. The user or a healthcare professional, such as a dermatologist, accesses and reviews these records remotely via a web interface or mobile application, enabling monitoring of treatment progress over time. The phototherapy device system receives software updates, new treatment protocols, or configuration data from the cloud server. Remote diagnostics, including battery level, system status, and error logs, are transmitted to the cloud for service and support purposes.

The phototherapy device system supports full offline functionality when internet connectivity is unavailable. The controller 112 stores the skin blemish recognition model, calibration data, treatment protocols, and user profiles locally in the memory 122, enabling complete operation without cloud access. Treatment sessions conducted offline are logged locally and automatically synchronized to the cloud when connectivity is restored. Offline mode is particularly useful for travel or in locations with limited internet access. The system also supports multi-device synchronization, allowing treatment data to be accessed across multiple devices owned by the user, such as smartphones, tablets, or computers, with data synchronized via the cloud when connectivity is available.

The phototherapy device system supports a remote professional prescription mode for telehealth-based dermatological care. A healthcare professional, such as a dermatologist, remotely accesses the user's skin condition data and treatment history through a secure professional portal or application. The dermatologist reviews facial skin map information, examines detected skin conditions, and prescribes customized treatment protocols specifying wavelength selection, intensity levels, treatment duration, and treatment frequency for each identified area. The prescribed treatment protocol is transmitted to the controller 112 via the cloud platform and subsequently downloaded to the user's phototherapy device system. The user then executes the prescribed treatment protocol, and treatment session data are uploaded back to the cloud for the dermatologist to review treatment compliance and outcomes. The professional mode includes authentication mechanisms to verify the credentials of healthcare professionals.

The phototherapy device system supports data export in multiple formats to facilitate interoperability with other health systems. Treatment history, progress images, and detected blemish data are exported from the companion mobile application as PDF reports for sharing with healthcare providers, CSV files for data analysis, or in standardized healthcare data formats such as FHIR for integration with electronic health record systems. The export function includes options to select date ranges, data types, and anonymization preferences. The system integrates with third-party health and wellness platforms such as Apple Health, Google Fit, or Samsung Health, allowing users to view their phototherapy activity alongside other health metrics. Integration with electronic health record systems used by dermatology clinics enables healthcare providers to access patient treatment data directly within their clinical workflow.

The phototherapy device system is accompanied by a companion mobile application that provides enhanced functionality and user engagement. The companion application is installed on a smartphone or tablet and communicates with the controller 112 via Bluetooth, Wi-Fi, or other wireless protocols. The application provides device pairing and configuration functions, allowing the user to customize treatment preferences, adjust default parameters, and manage multiple user profiles. The application displays real-time treatment status, including active zones, elapsed time, and remaining time. Push notifications inform the user of treatment milestones, encourage consistency, or alert them to software updates available for the phototherapy device system. The application provides a digital representation of the mask body 100 showing the selected treatment zones, allowing the user to visualize and confirm selections before treatment begins.

The phototherapy device system provides audio and voice feedback to enhance user awareness during treatment. Audible tones or voice prompts indicate treatment start, progress milestones such as halfway completion, and session end. Voice prompts announce detected blemish counts, selected treatment modes, remaining treatment time, or safety warnings. The audio feedback is delivered through a speaker integrated into the controller 112 or transmitted wirelessly to the user's smartphone or earbuds. Volume levels are adjustable, and users have the option to mute audio feedback if preferred. The phototherapy device system also includes a voice interface for hands-free operation, with a microphone and speech recognition module allowing the user to issue spoken commands such as “start treatment,” “pause session,” “increase intensity,” or “select red light” to control the phototherapy session without manually touching the mask body 100 or controller 112.

The phototherapy device system displays a treatment progress indicator and calculates an efficacy score to inform the user of treatment status and outcomes. The progress indicator is a visual display on the controller 112, illuminated segments on the mask body 100, or a graphical element in the companion mobile application showing elapsed time, remaining time, percentage complete, or a countdown timer. The efficacy score is computed based on changes in detected blemish count, total blemish area, average severity level, or other metrics derived from facial images captured before each session. The score is presented as a numerical value, percentage improvement, or graphical trend over the treatment course. Users view their efficacy score in the companion application to track progress and assess the effectiveness of their treatment regimen. In some cases, the efficacy score is shared with healthcare providers to support clinical decision-making.

During an active treatment session, the LED beads 108 in a treatment zone gradually change color or intensity as treatment progresses, providing a visual indication of elapsed treatment time directly on the mask body 100. For example, the LED beads 108 transition from a brighter intensity at the start of treatment to a dimmer intensity as treatment nears completion, or shift in color temperature to indicate progress. Alternatively or additionally, the controller 112 emits periodic audible signals, such as beeps at regular intervals to indicate treatment progress, or provides periodic haptic feedback through the haptic feedback mechanism via vibration pulses. A distinct audible or haptic pattern indicates when treatment is complete. These on-mask progress indicators help users track treatment status without needing to view a separate display.

The phototherapy device system employs predictive analytics to forecast treatment outcomes and provides personalized skincare recommendations. By analyzing the user's past treatment sessions, detected blemish progression, and skin response patterns stored in the user profile, the processor 120 predicts the expected time to achieve specified improvement milestones. The predictions are displayed to the user to set realistic expectations and encourage treatment adherence. The predictive model is trained on aggregated anonymized data from multiple users with similar skin types and conditions, improving prediction accuracy through population-level insights. Based on the detected blemish types, skin tone analysis, and treatment progress, the system suggests compatible topical skincare products such as cleansers, moisturizers, serums, or sunscreens that enhance therapeutic outcomes. In some cases, the system advises against products containing ingredients that interfere with phototherapy or increase photosensitivity.

The phototherapy device system includes treatment memory and condition location history functionality. The memory 122 stores records of previously selected treatment zones, detected skin conditions, and their locations from earlier treatment sessions. This historical data is particularly beneficial for users with chronic skin conditions who regularly treat the same facial areas. When initiating a new treatment session, the system presents the user with options to quickly re-select previously treated areas or to load a previous treatment configuration. The condition location history tracks changes in skin conditions over time at specific facial locations, enabling the user or a connected healthcare professional to monitor treatment progress for individual blemishes. The system identifies patterns in treatment selections and proactively suggests treatment zones based on historical usage patterns.

The phototherapy device system provides an augmented reality (AR) preview feature to help the user visualize treatment zones before donning the mask body 100. The user uses a smartphone or tablet running the companion mobile application to capture a photo or live video of their face. The application displays an overlay showing the detected blemish regions and proposed LED activation areas as a color-coded mask graphic superimposed on the user's face image. The user adjusts or confirms the treatment areas on the touchscreen before the mask session begins. Once the mask body 100 is worn, the controller 112 receives the mapped treatment zones from the application and automatically activates the corresponding LED beads 108. This AR-assisted mapping helps ensure that the targeted LED clusters align with the user's blemishes by confirming the treatment plan visually before wearing the mask body 100.

The phototherapy device system includes environmental sensors for automatic adaptation of treatment parameters based on ambient conditions. The environmental sensors include temperature sensors, humidity sensors, or UV index sensors integrated into the mask body 100, the controller 112, or a connected smart device. The phototherapy device system also includes a skin moisture or hydration sensor on the inner translucent bottom layer 110 to gauge the user's skin hydration level before or during therapy. If the skin moisture sensor detects very dry skin, the processor 120 reduces light intensity or suggests a soothing regimen. The processor 120 adjusts treatment parameters in response to detected environmental conditions to optimize treatment efficacy and safety. For example, in cold and dry conditions where ambient temperature is below a threshold, such as 5 degrees Celsius, and humidity is below a threshold, such as 20 percent, the system automatically reduces blue light exposure time to prevent excessive skin drying or increases red light exposure to promote skin hydration and regeneration. In high-humidity conditions, the system adjusts treatment intensity or duration accordingly. The environmental adaptation also accounts for geographic location and seasonal variations, with the system accessing weather data through a connected smart device to anticipate environmental conditions.

The phototherapy device system includes parental control features to ensure safe use by children or adolescents. Parental controls impose restrictions such as maximum treatment duration per session, maximum sessions per day or week, intensity limits, and restricted access to certain wavelengths. A parent or guardian configures these restrictions via the companion mobile application using a PIN or password. The system requires parental authorization before a child user can initiate treatment or modify settings beyond the permitted range. The parental control settings are associated with specific user profiles, allowing different restriction levels for different family members based on age and skin sensitivity.

The phototherapy device system implements data security and privacy protection measures to safeguard user information. Facial images and treatment data stored on the device or transmitted to cloud servers are encrypted using industry-standard encryption protocols to prevent unauthorized access. User authentication mechanisms such as password protection, biometric authentication, or secure login credentials are required before accessing stored treatment records or personal data. When data is transmitted wirelessly to cloud servers or external devices, the communication is secured using encrypted protocols such as TLS or SSL. The system implements data anonymization techniques when aggregating treatment data for analytics or research purposes, removing or obfuscating personally identifiable information. Users are provided with controls to manage their data, including options to view, export, or delete their stored facial images and treatment history.

The phototherapy device system includes error-handling mechanisms to manage various failure conditions gracefully. If the skin blemish recognition model fails to detect any blemishes in the captured facial image, the system prompts the user to recapture the image, adjust lighting conditions, or proceed with a default full-face treatment mode. If the calibration process fails to establish accurate face-to-mask mapping, the system alerts the user to reposition the mask body 100 and retry calibration, or falls back to a predefined default mapping. In cases where the imaging device malfunctions, the system notifies the user and offers manual zone selection via the touch-sensitive layer 102 as an alternative input method. If wireless communication with the cloud server fails during data upload, the system stores the session data locally in the memory 122 and retries transmission when connectivity is restored. The controller 112 maintains an error log that records detected faults, failed operations, and system warnings for diagnostic purposes.

The phototherapy device system incorporates a stretchable or elastic substrate construction to maintain touch-sensing accuracy during stretching and conforming to facial contours. The flexible light panel 106 comprises an elastic substrate made of materials such as dimethyl siloxane, polyimide, or flexible polymeric materials that provide elasticity while supporting the electronic components. The flexible light panel 106 includes solid material areas carrying the LED beads 108 and a plurality of openings between the solid areas, where the openings accommodate larger deformations when the mask body 100 is stretched, while the solid material areas experience smaller deformations to protect the LED beads 108 and associated circuitry. Touch-sensing elements of the touch-sensitive layer 102 are positioned such that their locations correspond to the solid material areas of the flexible light panel 106, ensuring that the touch-sensing elements remain protected and accurately positioned relative to the corresponding LED beads 108 even when the mask body 100 is stretched or flexed to conform to different facial shapes and sizes. The processor 120 firmware accounts for stretching of the mask body 100 when mapping raw touch coordinates from the touch-sensitive layer 102 to the LED beads 108 on the flexible light panel 106. The processor 120 applies geometric correction factors based on the known elastic properties of the mask body 100 materials and the current tension state detected by the fit sensors, ensuring that touch coordinates are accurately mapped to the corresponding LED beads 108 despite deformation of the mask body 100.

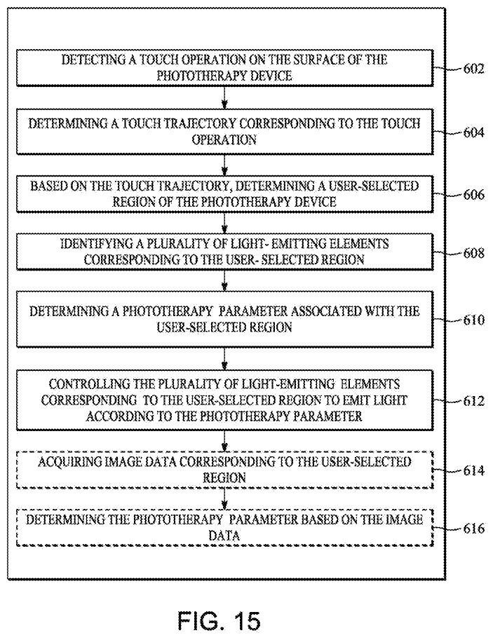

Referring to FIG. 15, a flow diagram of a method for operating a phototherapy device is illustrated. The method begins at step 602, where a touch operation on the surface of the phototherapy device is detected. The method proceeds to step 604, where a touch trajectory corresponding to the touch operation is determined. The method continues to step 606, where, based on the touch trajectory, a user-selected region of the phototherapy device is determined. At step 608, a plurality of light-emitting elements corresponding to the user-selected region are identified. At step 610, a phototherapy parameter associated with the user-selected region is determined. At step 612, the plurality of light-emitting elements corresponding to the user-selected region are controlled to emit light according to the phototherapy parameter. The method further includes step 614, where image data corresponding to the user-selected region is acquired, and step 616, where the phototherapy parameter is determined based on the image data. Steps 616 and 614 are performed after step 606 and before step 612 to enable AI-assisted determination of the phototherapy parameter for the user-selected region.

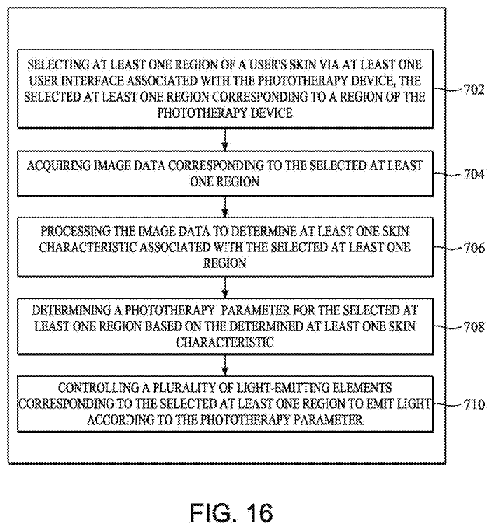

Referring to FIG. 16, a flow diagram of a method for operating a phototherapy device is illustrated. The method begins at step 702, where at least one region of a user's skin is selected via at least one user interface associated with the phototherapy device, the selected at least one region corresponding to a region of the phototherapy device. The method proceeds to step 704, where image data corresponding to the selected at least one region is acquired. The method continues to step 706, where the image data is processed to determine at least one skin characteristic associated with the selected at least one region. At step 708, a phototherapy parameter for the selected at least one region is determined based on the determined at least one determined skin characteristic. The method concludes at step 710, where a plurality of light-emitting elements corresponding to the selected at least one region are controlled to emit light according to the phototherapy parameter.

The phototherapy device system comprises one or more multi-modal stimulation components in addition to the LED beads 108 for phototherapy. The stimulation components include microcurrent electrodes configured to deliver low-level electrical stimulation to facial muscles, ultrasonic transducers configured to emit ultrasonic waves for enhanced product penetration or tissue stimulation, magneto-therapy components configured to deliver magnetic field therapy, Peltier components configured to provide thermoelectric heating or cooling, heating elements configured to provide controlled warmth to treatment areas, cooling elements configured to provide soothing cooling effects, or massage elements configured to provide mechanical stimulation. Each stimulation component operates independently or in combination with the phototherapy provided by the LED beads 108 to provide a multi-modal treatment experience. The processor 120 coordinates the activation of different stimulation components according to treatment protocols stored in the memory 122, enabling combination treatments that leverage multiple therapeutic modalities within a single session.

The phototherapy device system implements power conservation features to extend battery life and enable longer treatment sessions. By spatially limiting the phototherapy illumination to only user-selected areas or AI-detected blemish regions rather than activating all LED beads 108 simultaneously, the phototherapy device system consumes substantially less electrical power during treatment sessions compared to full-mask illumination. This selective activation approach enables the use of smaller and lighter batteries while maintaining adequate treatment duration or enables longer treatment sessions without requiring battery recharging. The processor 120 implements additional power-saving measures, such as reducing LED intensity when the mask body 100 is detected to be in close contact with the skin via the fit sensors, entering a low-power standby mode when no user input is detected for a predetermined period, or dimming indicator lights during active treatment to conserve power for the therapeutic LED beads 108.

The control program 124 stored in the memory 122 is embodied as program code in any suitable form, such as source code, object code, executable code, or an intermediate representation. A computer-readable storage medium carrying such program code includes, by way of non-limiting example, recording media, USB flash drives, portable hard disks, optical disks, magnetic disks, on-board memory, read-only memory (ROM), random access memory (RAM), solid-state drives, or other tangible storage devices. When the control program 124 is executed by the processor 120, the processor 120 implements the steps of the touch control method, the AI control method, or both, as described herein. The control program 124 is organized into one or more software modules or functional units stored in the memory 122 and executed by the processor 120 to realize the functions of the disclosed phototherapy device system.

The phototherapy device system supports region selection via a mobile application communicatively coupled to the phototherapy device. The companion mobile application displays a digital representation of the user's face or the mask body 100 on the external device screen, allowing the user to select treatment regions by tapping, drawing, or marking areas on the displayed representation. The selected regions are transmitted from the mobile application to the controller 112 via Bluetooth, Wi-Fi, or other wireless communication protocols. The controller 112 maps the selected regions to corresponding LED beads 108 on the flexible light panel 106 and activates the corresponding LED beads 108 according to the determined phototherapy parameters. This mobile application-based region selection provides an alternative to direct on-mask touch input, particularly when the user is wearing the mask body 100 and direct touch access to the touch-sensitive layer 102 may be limited.

A computer program product is provided that, when run on an electronic device, causes the electronic device to perform the steps of the touch control method, the AI control method, or both, as described herein. The electronic device comprises the memory 122, the processor 120, and the control program 124 stored in the memory 122 and executable on the processor 120. The electronic device is embodied in various hardware platforms, including the controller 112 with onboard control electronics, desktop computers, laptop computers, handheld devices such as tablets or smartphones, and server systems.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A phototherapy device system, comprising:
a touch-sensing module configured to detect a touch operation on a surface of a phototherapy device;
a trajectory determination module configured to determine a touch trajectory corresponding to the touch operation;
a mask area determination module configured to determine, based on the touch trajectory, a user-selected region of the phototherapy device;
a light-emitting control module configured to identify a plurality of light-emitting elements corresponding to the user-selected region;
a processing module configured to determine a phototherapy parameter associated with the user-selected region; and
the light-emitting control module configured to control the plurality of light-emitting elements corresponding to the user-selected region to emit light according to the phototherapy parameter.

2. The system of claim 1, wherein:
the touch trajectory comprises at least one of a single-point touch, an open trajectory, or a closed trajectory; and
the user-selected region comprises:
a point region corresponding to the single-point touch,
a path region corresponding to the open trajectory, or
an enclosed region corresponding to the closed trajectory.

3. The system of claim 1, wherein the touch-sensing module is further configured to determine a touch force associated with the touch operation.

4. The system of claim 3, wherein the mask area determination module is configured to determine a size of the user-selected region based on the touch force.

5. The system of claim 1, wherein the processing module is configured to determine the phototherapy parameter based on user input corresponding to a selected phototherapy mode.

6. The system of claim 1, wherein the processing module is further configured to:
acquire image data corresponding to the user-selected region; and
analyze the image data to identify at least one skin characteristic,
wherein the phototherapy parameter is determined based on the skin characteristic.

7. The system of claim 6, wherein:
the processing module is configured to determine a recommended phototherapy parameter based on the image data; and
the phototherapy parameter is determined based on user modification of the recommended phototherapy parameter.

8. The system of claim 1, wherein the light-emitting control module is configured to place the plurality of light-emitting elements in a standby state before emission of light based on the phototherapy parameter.

9. The system of claim 1, wherein: the phototherapy device comprises a plurality of touch-sensitive elements distributed across a surface of the phototherapy device, each touch-sensitive element being spatially aligned with a corresponding light-emitting element; each touch-sensitive element comprises at least one of a capacitive sensor, a resistive sensor, or a pressure sensor; and a touch detected by a given touch-sensitive element activates the corresponding light-emitting element to emit light according to the phototherapy parameter.

10. The system of claim 1, wherein: the phototherapy device comprises a touch-sensitive layer disposed on a surface of the phototherapy device; and the touch-sensitive layer is configured to detect a spatial location of a touch input and activate a corresponding subset of the plurality of light-emitting elements to emit light according to the phototherapy parameter.

11. The system of claim 6, wherein the processing module is configured to:
determine a severity level associated with the skin characteristic of the user-selected region, and
adjust the phototherapy parameter based on the severity level,
wherein the severity level is determined based on at least one of a size, intensity, density, or spatial distribution of the skin characteristic.

12. A method for operating a phototherapy device, comprising:
detecting a touch operation on a surface of the phototherapy device;
determining a touch trajectory corresponding to the touch operation;
determining, based on the touch trajectory, a user-selected region of the phototherapy device;
identifying a plurality of light-emitting elements corresponding to the user-selected region;
determining a phototherapy parameter associated with the user-selected region; and
controlling the plurality of light-emitting elements corresponding to the user-selected region to emit light according to the phototherapy parameter.

13. The method of claim 12, further comprising:
acquiring image data corresponding to the user-selected region; and
determining the phototherapy parameter based on the image data.

14. The method of claim 12, further comprising:
detecting an indication associated with the user-selected region, wherein the indication comprises at least one of a therapeutic substance, a color marking, a symbol, a pattern, or a visually or sensor-detectable marker; and
determining the phototherapy parameter based on the detected indication.

15. The method of claim 12, wherein determining the phototherapy parameter comprises:
determining the phototherapy parameter based on a type of finger used for the touch operation, and
determining an intensity of emitted light based on a pressure associated with the touch operation,
wherein different fingers correspond to different phototherapy parameters.

16. The method of claim 12, further comprising:
providing feedback indicating the user-selected region,
wherein the feedback comprises at least one of:
haptic feedback, including vibration generated by the phototherapy device, or
visual feedback, including illumination of one or more light-emitting elements,
and wherein the feedback is provided on at least one of an outer surface or an inner surface of the phototherapy device corresponding to the user-selected region.

17. The method of claim 12, wherein determining the user-selected region comprises mapping the touch trajectory to a spatial distribution of the plurality of light-emitting elements on a curved surface of the phototherapy device.

18. A method for operating a phototherapy device, comprising:
selecting at least one region of a user's skin via at least one user interface associated with the phototherapy device, the selected at least one region corresponding to a region of the phototherapy device;
acquiring image data corresponding to the selected at least one region;
processing the image data to determine at least one skin characteristic associated with the selected at least one region;
determining a phototherapy parameter for the selected at least one region based on the at least one skin characteristic; and
controlling a plurality of light-emitting elements corresponding to the selected at least one region to emit light according to the phototherapy parameter.

19. The method of claim 18, wherein selecting the at least one region comprises receiving a touch input on a surface of the phototherapy device.

20. The method of claim 18, wherein selecting the at least one region comprises selecting the at least one region from a visual representation of the user's skin displayed on an external device.

21. The method of claim 20, wherein:
the external device comprises at least one of a mobile device or a smart mirror; and
a region selection input comprises at least one symbol corresponding to a selected region of the user's skin, and
the phototherapy parameter is assigned to the selected region based on the symbol.

22. The method of claim 18, wherein:
selecting the at least one region comprises selecting two or more regions associated with different skin characteristics;
respective phototherapy parameters correspond to different wavelengths or light characteristics; and
the plurality of light-emitting elements is controlled to simultaneously emit different lights in the respective regions.

23. The method of claim 18, wherein:
processing the image data comprises generating a recommended phototherapy parameter based on the image data; and
the phototherapy parameter is determined based on at least one of user confirmation or user modification of the recommended phototherapy parameter.

24. A phototherapy device, comprising:
an image acquisition module configured to acquire image data of a user;
a processing module configured to:
identify at least one skin region and a corresponding skin characteristic from the image data, and
determine a phototherapy parameter based on the skin characteristic; and
a light-emitting control module configured to control a plurality of light-emitting elements corresponding to the at least one skin region to emit light according to the phototherapy parameter.

25. The device of claim 24, wherein:
the processing module is further configured to identify the at least one skin region based on a user input received via at least one of:
a touch input on the phototherapy device, or
an input received from an external device.

26. The device of claim 24, wherein the processing module is configured to:
determine a capture angle of the image data; and
generate corrected image data based on the capture angle before identifying the at least one skin region.

27. The device of claim 24, wherein:
the processing module is configured to determine a severity level associated with the skin characteristic, and
the severity level is determined based on at least one of a size, intensity, density, or spatial distribution of the skin characteristic.

28. The device of claim 24, wherein:
the system further comprises a memory configured to store historical phototherapy data, and the processing module is configured to:
compare current image data with the historical phototherapy data to determine a treatment effect, and
adjust the phototherapy parameter based on the treatment effect.
